(19) United States
(12) Patent Application Publication
(10) Pub. No.: US 2025/0319068 A1
(43) Pub. Date: Oct. 16, 2025

(54) TREATMENT OF DISEASES VIA ADMINISTRATION OF BUNTANETAP AND AN ANTIHYPERTENSIVE AGENT

(71) Applicant: ANNOVIS BIO, INC., Berwyn, PA (US)

(72) Inventor: Maria Maccecchini, West Chester, PA (US)

(73) Assignee: ANNOVIS BIO, INC., Berwyn, PA (US)

(21) Appl. No.: 18/899,948

(22) Filed: Sep. 27, 2024

Related U.S. Application Data
(60) Provisional application No. 63/633,912, filed on Apr. 15, 2024.

(51) Int. Cl. A61K 31/407; A61K 31/517; A61P 25/28

(52) U.S. Cl. CPC A61K 31/407; A61K 31/517; A61P 25/28

(57) ABSTRACT

The invention relates to methods and pharmaceutical compositions effective for treating, inhibiting, preventing, slowing, or delaying the onset of a neurodegenerative disease in mammals (e.g., humans) via the co-administration of an effective amount of a compound selected from the group consisting of Formula (I), Formula (II), Formula (III) or Formula (IV) or pharmaceutically acceptable salts thereof and an antihypertensive drug (an alpha-adrenergic blocker). In certain embodiments, the mammal is a healthy human, or a human experiencing cognitive dysfunction with or without hypertension.

This application claims the benefit of U.S. Provisional Application No. 63/633,912, filed on Apr. 15, 2024, hereby incorporated by reference.

### FIELD OF THE INVENTION

The present patent application concerns a method of treating neurodegenerative diseases, cardiovascular diseases and benign prostatic hyperplasia via administration of buntanetap or related compounds, together with an antihypertensive agent (e.g., an alpha-adrenergic blocker).

### BACKGROUND OF THE INVENTION

High iron levels increase the translation of neurotoxic aggregating proteins, leading, e.g., to impairment of axonal transport, inflammation, nerve cell death, and cognitive and motor function impairments.

For unknown reasons, in a “sick” brain, the level of iron is high, which induces iron regulatory protein 1 to release mRNAs coding for neurotoxic aggregating proteins causing upregulation of translation and synthesis, leading to overproduction of neurotoxic aggregating proteins in the sick brain.

Abeta is a well-known contributor to the development of Alzheimer's disease. Abeta plaques are found throughout the brains of people who develop the cognitive symptoms of Alzheimer's disease.

Dementia with Lewy bodies is the second most common neurodegenerative form of dementia after Alzheimer's disease. Dementia with Lewy bodies impacts memory, movement, and cognition.

Buntanetap is a translational inhibitor of multiple neurotoxic aggregating proteins.

Recent studies have revealed that cardiovascular diseases including high blood pressure are risk factors for cognitive decline and dementia through cerebral perfusion damage, brain structural changes, inflammation, β-amyloid deposition, and neuroendocrine disorders. See, e.g., Morley J E. Cognition and chronic disease. J Am Med Dir Assoc 2017; 18:369-371; Greaves D, Psaltis P J, Ross T J, et al. Cognitive outcomes following coronary artery bypass grafting: a systematic review and meta-analysis of 91,829 patients. Int J Cardiol 2019; 289:43-49; Cardiogenic dementia. Lancet 1977; 309:27-28; Leng X, Espeland M A, Manson J E, et al. Cognitive function, and changes in cognitive function as predictors of incident cardiovascular disease: the Women's Health Initiative Memory Study. J Gerontol A Biol Sci Med Sci 2018; 73:779-785; Ewcharoen J, Trongtorsak A, Kanitsoraphan C, et al. Cognitive impairment, and 30-day rehospitalization rate in patients with acute heart failure: a systematic review and meta-analysis. Indian Heart J 2019; 71:52-59; Irimata K E, Dugger B N, Wilson J R. Impact of the presence of select cardiovascular risk factors on cognitive changes among dementia subtypes. Curr Alzheimer Res 2018; 15:1032-1044; Yuan J Q, Lv Y B, Chen H S, et al. Association between late-life blood pressure and the incidence of cognitive impairment: a community-based prospective cohort study. J Am Med Dir Assoc 2019; 20:177-182.e172; Irimata K E, Dugger B N, Wilson J R. Impact of the presence of select cardiovascular risk factors on cognitive changes among dementia subtypes. Curr Alzheimer Res 2018; 15:1032-1044; Jefferson A L. Cardiac output as a potential risk factor for abnormal brain aging. J Alzheimer's Dis 2010; 20:813-821; Stefanidis K B, Askew C D, Greaves K, et al. The effect of non-stroke cardiovascular disease states on risk for cognitive decline and dementia: a systematic and meta-analytic review. Neuropsychol Rev 2018; 28:1-15; Haring B, Leng X, Robinson J, Johnson K C, Jackson R D, Beyth R, Wactawski-Wende J, von Ballmoos M W, Goveas J S, Kuller L H, Wassertheil-Smoller S. Cardiovascular disease and cognitive decline in postmenopausal women: results from the Woman's Health Initiative Memory Study, J Am Heart Assoc. 2013 Dec. 18; 2(6): e000369.

Antihypertensive agents are not currently approved by U S FDA for treatment of neurodegenerative diseases.

### SUMMARY OF THE INVENTION

It is an object of the invention to provide a treatment for neurodegenerative diseases.

It is also an object of the invention to provide a treatment for cardiovascular diseases.

It is an additional object of the invention to provide a treatment for benign prostatic hyperplasia.

It is a further object of the invention to prevent, slow, or delay the development of neurodegenerative diseases that result in neuronal cell death.

In accordance with the above objects and others, the invention is directed in part to a method of treating a neurodegenerative disease via the administration of buntanetap, a compound that is similar to buntanetap as described herein, pharmaceutically acceptable salts and complexes thereof, together with an antihypertensive drug (e.g., an alpha-adrenergic blocker), wherein an administered amount of at least one of these agents is at least 2 times lower than the amount of that agent that would be required to provide any meaningful therapeutic effect if that agent were to be administered alone (i.e., as a monotherapy, without the other agent). Yet, the therapeutic effect provided by administration of (i) buntanetap, or the compound that is similar to buntanetap, together with (ii) the antihypertensive agent is (a) greater than the therapeutic effect, if any, provided by administration of the same amount of buntanetap, or the compound that is similar to buntanetap, alone, and (b) is greater than the therapeutic effect, if any, provided by administration of the same amount of the antihypertensive agent alone. In the methods of the invention, buntanetap, or the compound that is similar to buntanetap, and the antihypertensive agent may be administered in the same or different dosage form(s). In addition to the buntanetap and the antihypertensive agent, the dosage form may comprise one or more pharmaceutically acceptable excipients.

The invention is also directed in part to a method of treating a disease via administration of (i) buntanetap, or a compound that is similar to buntanetap, together with (ii) an antihypertensive agent to a human in need thereof, wherein the buntanetap and the antihypertensive agent are administered in amounts that are synergistic.

The invention is further directed to a method of treating a disease via the administration of an amount of buntanetap, or the compound that is similar to buntanetap, together with an amount of an antihypertensive agent to a human patient in need thereof, wherein the amount of the antihypertensive agent is subtherapeutic when administered without buntanetap, or the compound that is similar to buntanetap, but is therapeutic when administered with the amount of buntanetap, or the compound that is similar, to buntanetap.

The invention is also directed to a method of treating a disease via the administration of an amount of buntanetap, or the compound that is similar to buntanetap, together with an amount of an antihypertensive agent to a human patient in need thereof, wherein the amount of buntanetap, or the compound that is similar to buntanetap, is subtherapeutic when administered without the antihypertensive agent, but is therapeutic when administered with the amount of the antihypertensive agent.

The diseases amenable to the treatment in accordance with the present invention include, e.g., neurodegenerative diseases, cardiovascular diseases and benign prostatic hyperplasia.

The invention is further directed to a method of treating a neurodegenerative disease via the administration of an amount of buntanetap, or the compound that is similar to buntanetap, together with an amount of an antihypertensive agent to a human patient in need thereof, wherein the amount of the antihypertensive agent is subtherapeutic when administered without buntanetap, or the compound that is similar to buntanetap, but is therapeutic when administered with the amount of buntanetap, or the compound that is similar, to buntanetap, wherein the antihypertensive agent is an alpha-1 antagonist.

The invention is also directed to a method of treating a disease via the administration of an amount of buntanetap, or the compound that is similar to buntanetap, together with an amount of an antihypertensive agent to a human patient in need thereof, wherein the amount of buntanetap, or the compound that is similar to buntanetap, is subtherapeutic when administered without the antihypertensive agent, but is therapeutic when administered with the amount of the antihypertensive agent, wherein the antihypertensive agent is an alpha-1 antagonist.

The administration of (i) buntanetap, or a compound that is similar to buntanetap, together with (ii) an antihypertensive agent may provide a therapeutic effect that is at least 4 times greater than the therapeutic provided by administration of the antihypertensive agent alone. In certain embodiments, improvement provided by administration of buntanetap, or the compound that is similar to buntanetap, together with the antihypertensive agent is 5 to 10 times greater than the improvement provided by administration of the antihypertensive agent alone.

The invention encompasses a method of treating a disease via administration of (i) buntanetap, or a compound that is similar to buntanetap as described herein, together with (ii) an antihypertensive agent to a human in need thereof, wherein an administered amount of at least one of these agents is at least 5, 8 or 10 times lower than the amount of that agent that would be required to provide any meaningful therapeutic effect if that agent were to be administered alone.

The invention encompasses a method of treating a disease via administration of (i) buntanetap, or a compound that is similar to buntanetap as described herein, together with (ii) an alpha-1 antagonist to a human in need thereof, wherein an administered amount of at least one of these agents is at least 5, 8 or 10 times lower than the amount of that agent that would be required to provide any meaningful therapeutic effect if that agent were to be administered alone.

The methods of the present invention may therefore allow for (i) administration of (a) a lower dose of buntanetap, or the compound that is similar to buntanetap, and/or (b) a lower dose of the antihypertensive agent (e.g., an alpha-1 antagonist), (ii) a reduction in incidence and/or severity of adverse effect(s), and/or (iii) an increased efficacy of (a) the buntanetap, or the compound that is similar to buntanetap, and/or (b) the antihypertensive agent.

For example, in some of the embodiments, the invention is directed to a method of treating a disease via administration of (i) buntanetap, or a compound that is similar to buntanetap as described herein, together with (ii) an antihypertensive agent to a human in need thereof, wherein buntanetap, or the compound that is similar to buntanetap, is administered in an amount that per day is less than 1 mg, less than 0.9 mg or less than 0.8 mg. In some of these embodiments, the administered amount of buntanetap, or the compound that is similar to buntanetap, may e.g., be from about 0.01 mg to about 0.7 mg, and is administered orally, parenterally or transdermally.

The invention is also directed in part to a method of treating a disease via administration of (i) buntanetap, or a compound that is similar to buntanetap as described herein, together with (ii) an antihypertensive agent to a human in need thereof, wherein the antihypertensive agent is administered in an amount that per day is less than 1 mg, less than 0.5 mg or less than 0.1 mg. In some of these embodiments, the administered amount of the antihypertensive agent may e.g., be from about 0.01 mg to about 0.5 mg, and is administered orally, parenterally or transdermally.

The invention is also directed in part to a method of treating a disease via administration of (i) buntanetap, or a compound that is similar to buntanetap as described herein, together with (ii) an antihypertensive agent to a human in need thereof, wherein the antihypertensive agent is administered in an amount that is lower than an amount of buntanetap, or the compound that is similar to buntanetap.

In certain embodiments, the antihypertensive agent is not a phosphodiesterase inhibitor.

Diseases that could be treated by the methods of the invention include, e.g., neurodegenerative diseases (e.g., Alzheimer's disease, dementia with Lewy bodies, frontotemporal dementia, other forms of dementia, chronic traumatic encephalopathy, tauopathies, alpha-synucleopathies, Prion's disease, Down Syndrome, Huntington's disease, multiple sclerosis, Amyloid Lateral Sclerosis), dementias, Parkinson's disease, cardiovascular disease, and benign prostatic hyperplasia.

Depending on the disease being treated, the therapeutic effect provided by the administration in accordance with the methods of the invention may include, e.g., a reduction of and/or severity of a symptom of a neurodegenerative disease, an improvement in cognitive function, an improvement in motor function, a reduction in blood pressure, and a reduction of a symptom of benign prostatic hyperplasia.

The invention is directed in part to a method of treating a neurodegenerative disease via the administration of (i) buntanetap, or the compound that is similar to buntanetap, together with (ii) an antihypertensive agent to a human patient in need thereof, wherein the neurodegenerative disease is Alzheimer's disease, and the therapeutic effect is an improvement in mental status.

The invention is also directed in part to a method of treating a neurodegenerative disease via the administration of (i) buntanetap, or the compound that is similar to buntanetap, together with (ii) an antihypertensive agent to a human patient in need, wherein the neurodegenerative disease is dementia (e.g., Parkinson's dementia, dementia with Lewy bodies, frontotemporal dementia, mild cognitive impairment, etc.), and the therapeutic effect is an improvement in cognition and mental status.

The improvements in mental status may, e.g., be measured by a change in a score on a cognitive scale (e.g., ADAS-Cog11).

The invention is also directed in part to a method of treating a neurodegenerative disease via the administration of (i) buntanetap, or the compound that is similar to buntanetap, together with (ii) an antihypertensive agent to a human patient in need thereof, wherein the neurodegenerative disease is Parkinson's disease, and the therapeutic effect is an improvement in motor function. The improvement in motor function may be measured, e.g., by a change in a score on MDS-UPDRS or a part thereof.

The invention is also directed in part to a method of treating Parkinson's disease via the administration of (i) buntanetap, or the compound that is similar to buntanetap, together with (ii) an antihypertensive agent to a human patient in need, wherein the therapeutic effect are improvements e.g. in Postural Instability, Gait Difficulty, Tremors, or Cognition.

The invention is also directed in part to a method of treating benign prostatic hyperplasia via the administration of (i) buntanetap, or the compound that is similar to buntanetap, together with (ii) an antihypertensive agent (e.g., an alpha-1 antagonist) to a human patient in need thereof. The improvement in benign prostatic hyperplasia may be measured, e.g., by a change in an International Prostate Symptom Score.

The invention is further directed in part to a method to inhibit, prevent or treat a neurodegenerative disease such as Alzheimer's disease via the administration of buntanetap, a compound that is similar to buntanetap as described herein, pharmaceutically acceptable salts and complexes thereof, together with an alpha-adrenergic blocker, and one or more pharmaceutically acceptable excipients, wherein the alpha-adrenergic blocker is alpha-1 antagonist.

The invention is further directed in part to a method to inhibit, prevent or treat a neurodegenerative disease such as Alzheimer's disease in a patient who has high blood pressure via the administration of buntanetap, compounds that are similar to buntanetap as described herein, pharmaceutically acceptable salts and complexes thereof, together with an antihypertensive agent.

The invention is further directed in part to a method of treating a disease via the administration of buntanetap, or the compound that is similar to buntanetap, together with an antihypertensive agent to a human patient in need thereof in a weight ratio that provides a synergistic therapeutic effect, as compared to the administration of buntanetap without the antihypertensive agent and/or administration of the antihypertensive agent without buntanetap.

The invention is also directed in part to part to a method of treating a disease via the administration of an amount of buntanetap, or the compound that is similar to buntanetap, together with an amount of an antihypertensive agent to a human patient in need thereof, wherein the amount of the antihypertensive agent is subtherapeutic when administered without buntanetap or the compound that is similar to buntanetap, but is therapeutic when administered with the amount of buntanetap or the compound that is similar to buntanetap.

The invention is also directed in part to part to a method of treating a disease via the administration of an amount of buntanetap, or the compound that is similar to buntanetap, together with an amount of an antihypertensive agent to a human patient in need thereof, wherein the amount of buntanetap or the compound that is similar to buntanetap is subtherapeutic when administered without the antihypertensive agent, but is therapeutic when administered with the amount of the antihypertensive agent.

The invention is also directed in part to a method of treating a dementia via the administration of buntanetap, or the compound that are similar to buntanetap, together with an antihypertensive agent to a human patient in need thereof in amounts that provide a synergistic therapeutic effect, as compared to the administration of buntanetap without the antihypertensive agent and/or administration of the antihypertensive agent without buntanetap.

The invention is also directed in part to a method of treating Alzheimer's disease via the administration of buntanetap, or the compound that is similar to buntanetap, together with an antihypertensive agent to a human patient in need thereof in amounts that provide a synergistic therapeutic effect, as compared to the administration of buntanetap without the antihypertensive agent and/or administration of the antihypertensive agent without buntanetap.

The invention is also directed in part to a method of treating a dementia with Lewy bodies via the administration of buntanetap, or the compound that are similar to buntanetap, together with an antihypertensive agent to a human patient in need thereof in amounts that provide a synergistic therapeutic effect, as compared to the administration of buntanetap without the antihypertensive agent and/or administration of the antihypertensive agent without buntanetap.

The invention is also directed in part to a method of treating Parkinson's disease via the administration of buntanetap, or the compound that is similar to buntanetap, together with an antihypertensive agent to a human patient in need thereof in amounts that provide a synergistic therapeutic effect, as compared to the administration of buntanetap without the antihypertensive agent and/or administration of the antihypertensive agent without buntanetap.

The invention is further directed in part to a method to inhibit, prevent or treat a neurodegenerative disease such as an alpha-synucleopathy via the administration of buntanetap, or the compound that is similar to buntanetap, together with an anti-hypertensive agent, and one or more pharmaceutically acceptable excipients.

The invention is further directed in part to a method to inhibit, prevent or treat a neurodegenerative disease such as Prion's disease via the administration of buntanetap, or the compound that is similar to buntanetap, together with an anti-hypertensive agent, and one or more pharmaceutically acceptable excipients.

The invention is further directed in part to a method to inhibit, prevent or treat a neurodegenerative disease such as Down Syndrome via the administration of buntanetap, or the compound that is similar to buntanetap, together with an anti-hypertensive agent, and one or more pharmaceutically acceptable excipients.

The invention is further directed in part to a method to inhibit, prevent or treat a neurodegenerative disease such as Huntington's disease via the administration of buntanetap, or the compound that is similar to buntanetap, together with an anti-hypertensive agent, and one or more pharmaceutically acceptable excipients.

The invention is further directed in part to a method to inhibit, prevent or treat a neurodegenerative disease such as Amyloid Lateral Sclerosis via the administration of buntanetap, or the compound that is similar to buntanetap, together with an anti-hypertensive agent, and one or more pharmaceutically acceptable excipients.

It is a further directed to a method to inhibit, prevent, slow or reduce the overexpression of neurotoxic aggregating proteins in a patient who is experiencing hypertension, who is not demonstrating symptoms of a neurological disorder or a neurodegenerative disease, or who is demonstrating symptoms of a neurological disorder or a neurodegenerative disease, comprising or consisting of administering to the human a therapeutically effective amount of buntanetap, active metabolites of buntanetap, therapeutically effective analogues of buntanetap, compounds that are similar to buntanetap as described herein, pharmaceutically acceptable salts and complexes thereof, together with a blood pressure medication in a therapeutic dose used to treat hypertension or in a subtherapeutic dose as compared to a dose of the blood pressure medication used to treat hypertension. Examples of neurotoxic aggregating proteins susceptible to treatment by the methods of the invention, include APP (amyloid precursor protein), Aβ (amyloid-β peptide, a fragment of APP), SOD (super oxide dismutase) proteins, Tau, alpha-synuclein (SNCA), transmissible spongiform encephalopathy (TSE) prions, TDP43, and huntingtin (HTT).

The invention is also directed in part to a method to treat, inhibit, or slow the onset of neurological disorders or diseases such as Alzheimer's disease, dementia with Lewy bodies, frontotemporal dementia, other dementias, chronic traumatic encephalopathy, tauopathies, Parkinson's disease, alpha-synucleopathies, Prion's disease, Down Syndrome, Huntington's disease, Amyloid Lateral Sclerosis, multiple sclerosis, other neurodegenerative diseases which present as misfolding, aggregation and accumulation of proteins in the brain, resulting in inflammation and eventual cell death, and hypertension in mammals (e.g., humans) in need thereof comprising or consisting of administering to the human a therapeutically effective amount of buntanetap, active metabolites of buntanetap, therapeutically effective analogues of buntanetap, compounds that are similar to buntanetap as described herein, pharmaceutically acceptable salts and complexes thereof, together with a blood pressure medication, and one or more pharmaceutically acceptable excipients.

The invention is further directed in part to a method for preventing, treating, inhibiting, or slowing a neurodegenerative disease comprising administering to a human in need thereof an antihypertensive agent together with a compound selected from the group consisting of Formula (I), Formula (II), Formula (III) and Formula (IV):

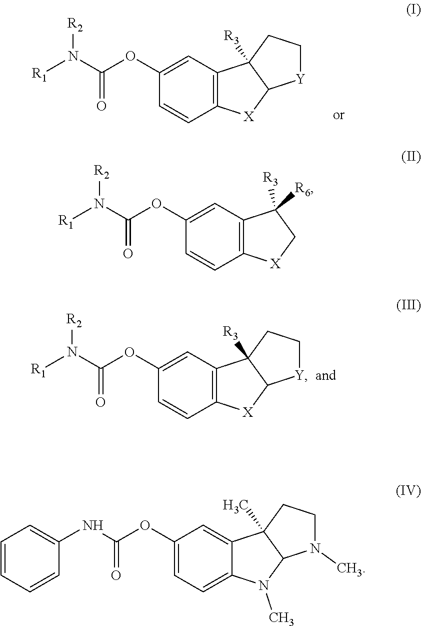

The invention is further directed in part to a method for treating a healthy human who is at risk of developing such a neurodegenerative disease comprising administering to the healthy human an alpha-adrenergic blocker together with a compound selected from the group consisting of Formula (I), Formula (II), Formula (III) and Formula (IV):

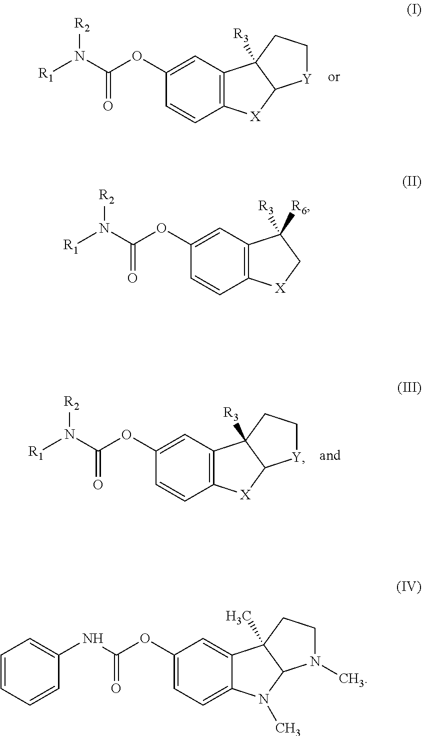

In Formula (I) and Formula (II), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen, C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4. In certain embodiments, the method includes the use of a second therapeutic active agent to treat, prevent, delay, or slow the onset of neurodegenerative disease such as Alzheimer's disease.

In certain embodiments, the compound selected from the group consisting of Formula (I), Formula (II), Formula (III) and Formula (IV) is devoid of any significant acetylcholinesterase (ACHE) inhibitory activity and devoid of any butyrylcholinesterase (BCHE) activity.

In Formula (III), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl.

In certain embodiments, the compound of Formula (I) and Formula (II) is the a (+)-enantiomer. In other embodiments, the compound of Formula (I) and Formula (II) is a (−)-enantiomer.

In certain embodiments, the compound of Formula (I) is buntanetap and has the following structure:

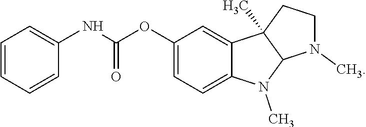 [SMILES: CN1CC[C@]2(C)c3cc(OC(=O)Nc4ccccc4)ccc3N(C)C12]

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 0.9 mg together with a therapeutically effective amount of an antihypertensive agent, to the human in need thereof for at least three months, and the administration results in an increase in a score on a cognitive test in the human.

In the methods of the invention, buntanetap, or the compound that is similar to buntanetap, and the antihypertensive agent may be administered as free bases, pharmaceutically acceptable salts, metabolites, prodrugs, complexes, and mixtures thereof by the same or different route of administration and at the same or different times.

The invention is also directed in part to a method to inhibit, prevent or treat a neurodegenerative disease via the administration of buntanetap, a compound that is similar to buntanetap as described herein, pharmaceutically acceptable salts and complexes thereof, together with an antihypertensive agent to a human patient in need thereof, wherein the buntanetap, or the compound that is similar to buntanetap, potentiates a therapeutic effect of the antihypertensive agent, and/or the antihypertensive agent potentiates a therapeutic effect of buntanetap, or the compound that is similar to buntanetap by at least 50% (e.g., by 100 to 900%).

The invention is also directed in part to a method of treating Alzheimer's disease via the administration of buntanetap, a compound that is similar to buntanetap as described herein, pharmaceutically acceptable salts and complexes thereof, together with an antihypertensive agent to a human patient in need thereof, wherein the buntanetap, or the compound that is similar to buntanetap, potentiates a therapeutic effect of the antihypertensive agent, and/or the antihypertensive agent potentiates a therapeutic effect of buntanetap, or the compound that is similar to buntanetap by at least 50% (e.g., by 100 to 900%).

The invention is also directed in part to a method to inhibit, prevent or treat a neurodegenerative disease via the administration of buntanetap, a compound that is similar to buntanetap as described herein, pharmaceutically acceptable salts and complexes thereof, together with an alpha-1 antagonist to a human patient in need thereof, wherein the buntanetap, or the compound that is similar to buntanetap, potentiates a therapeutic effect of the alpha-1 antagonist, and/or the alpha-1 antagonist potentiates a therapeutic effect of buntanetap, or the compound that is similar to buntanetap by at least 50% (e.g., by 100 to 900%).

The invention is also directed in part to a method of treating benign prostatic hyperplasia via the administration of buntanetap, a compound that is similar to buntanetap as described herein, pharmaceutically acceptable salts and complexes thereof, together an antihypertensive agent to a human patient in need thereof, wherein the buntanetap, or the compound that is similar to buntanetap, potentiates a therapeutic effect of the anihypertensive agent, and/or the antihypertensive agent potentiates a therapeutic effect of buntanetap, or the compound that is similar to buntanetap by at least 50% (e.g., by 100 to 900%).

The invention is further directed in part to a method of treating a disease via the administration of buntanetap, or the compound that is similar to buntanetap, together with an antihypertensive agent to a human patient in need thereof in a weight ratio that provides a synergistic therapeutic effect, as compared to the administration of buntanetap without the antihypertensive agent and/or administration of the antihypertensive agent without buntanetap. In some of the embodiments, the synergistic ratio weight ratio is from 10 to 10000, from 10 to 5000, from 10 to 1000, from 10 to 500, or from 10 to 250.

The invention is also directed in part to a method of treating a cardiovascular disease via the administration of buntanetap, or the compound that is similar to buntanetap, together with an antihypertensive agent to a human patient in need thereof in amounts that provide a synergistic therapeutic effect, as compared to the administration of buntanetap without the antihypertensive agent and/or administration of the antihypertensive agent without buntanetap.

In certain embodiments, buntanetap is administered in an amount from about 0.01 to about 120 mg, and numbers in between these numbers, preferably on a once-a-day basis. Thus, in certain embodiments, a dose of buntanetap may, e.g., be 0.01 mg, 0.02 mg, 0.05 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg, and numbers in between these numbers. In certain preferred embodiments, buntanetap is administered orally in a dose from about 0.1 mg to about 5 mg, from about 0.1 mg to about 2 mg or from about 0.1 mg to about 0.9 mg. In other preferred embodiments, the buntanetap dose is administered intravenously in an amount from about 0.01 to about 0.25 mg/day. In other preferred embodiments, the buntanetap dose is administered intraperitoneally/intramuscularly (IP/IM) in a dose from about 00.3 mg/day to about 0.7 mg/day. In certain preferred embodiments, the buntanetap is administered together with any antihypertensive agent as described further herein in following paragraphs.

In certain embodiments, buntanetap is administered orally in an amount from about 0.01 mg to about 0.9 mg, once-a-day or twice-a-day, and the antihypertensive agent is administered in an amount from about 0.1 mg to about 750 mg a once-a-day or twice-a-day.

In certain embodiments, buntanetap is administered orally in an amount from about 0.01 mg to about 0.9 mg, once-a-day or twice-a-day, and the antihypertensive agent is an alpha-1 antagonist and is administered once-a-day or twice-a-day in an amount from about 0.1 mg to about 50 mg.

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 0.9 mg together with a therapeutically effective amount of an alpha-1 antagonist, wherein the alpha-1 antagonist is selected from a group consisting of terazosin, doxazosin, alfuzosin, and pharmaceutically acceptable salts of any of the foregoing.

In certain embodiments, buntanetap is administered orally in a dose from about, e.g., 2 mg to about 80 mg, from about 3 mg to about 60 mg or from about 5 mg to about 30 mg. In other embodiments, the buntanetap dose is administered intravenously in an amount from about 0.1 to about 25 mg/day. In other embodiments, the buntanetap dose is administered intraperitoneally/intramuscularly (IP/IM) in a dose from about 0.3 mg/day to about 70 mg/day.

In certain embodiments of each of the methods described above, the oral pharmaceutical composition includes from about 0.1 mg to about 5 mg buntanetap or a pharmaceutically acceptable salt thereof, the IP/IM pharmaceutical composition includes from about 0.03 to about 0.7 mg buntanetap or a pharmaceutically acceptable salt thereof, and the intravenous (IV) pharmaceutical formulation includes from about 0.01 to about 0.25 mg buntanetap or a pharmaceutically acceptable salt thereof.

In certain embodiments of each of the methods described above, to minimize incidence of side effects, buntanetap or a pharmaceutically acceptable salt thereof is administered multiple times during the day or in a controlled or sustained release formulation that releases buntanetap or a pharmaceutically acceptable salt thereof over 4 to 24 hours, preferably over 8 to 24 hours or 12 to 24 hours.

In certain preferred embodiments of the methods described herein, peak plasma circulating levels of buntanetap in humans range, e.g., from about 0.01 ng/ml to about 200 ng/ml, in certain embodiments from about 0.2 ng/mL to about 20 ng/mL, and more preferably from about 0.37 ng/mL to about 12 ng/mL, and any numbers or integers in between. In certain preferred embodiments, the peak plasma circulating level is reached within about 1 to 3 hours after administration of buntanetap to humans. In certain embodiments, the steady-state plasma concentration of buntanetap in brain is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 ng/mL. In certain embodiments, the half-life of buntanetap in cerebrospinal fluid after administering is about 12 hours, and the half-life of buntanetap in plasma after administering is about 5 hours. In certain embodiments, the administration of buntanetap to humans results in a brain level of buntanetap that range from about 4 to about 10 times the plasma level of buntanetap in those patients. In certain embodiments, the concentration of buntanetap in the brain of humans is from about 0.8 ng/g to about 304 ng/g, in certain embodiments from about 0.3 ng/g to about 100 ng/g.

With respect to each of the methods described above, buntanetap, a pharmaceutically acceptable salt thereof, analogues or similar compounds as described herein may be administered, e.g., orally, parenterally, sublingually, via suppository, nasally, topically, transdermally, or via implant under the skin.

In certain embodiments, the antihypertensive agent is administered in an amount from about 0.01 to about 750 mg, and any numbers in between these numbers.

With respect to each of the methods described above, buntanetap, a pharmaceutically acceptable salt thereof, analogues or similar compounds as described herein may be administered, e.g., orally, parenterally, sublingually, via suppository, nasally, topically, transdermally, or via implant under the skin.

The invention is also directed in part to a method for treating, preventing, delaying, or slowing a neurodegenerative disease such a dementia comprising administering to the human a compound selected from the group consisting of Formula (I), Formula (II), Formula (III) or Formula (IV) together with an alpha-adrenergic blocker.

The invention is further directed in part to a method for treating, preventing, delaying, or slowing a neurodegenerative disease such as Alzheimer's disease comprising administering to the human a compound selected from the group consisting of Formula (I), Formula (II), Formula (III) or Formula (IV) together with an alpha-adrenergic blocker.

The invention is further directed in part to a method for treating, preventing, delaying, or slowing a neurodegenerative disease such as dementia with Lewy bodies comprising administering to the human a compound selected from the group consisting of Formula (I), Formula (II), Formula (III) or Formula (IV) together with an alpha-adrenergic blocker.

The invention is further directed in part to a method for treating, preventing, delaying, or slowing a neurodegenerative disease such as an alpha-synucleopathy comprising administering to the human a compound selected from the group consisting of Formula (I), Formula (II), Formula (III) or Formula (IV) together with alpha-adrenergic blocker.

The invention is further directed in part to a method for treating, preventing, delaying, or slowing a neurodegenerative disease such as an alpha-synucleopathy comprising administering to the human a compound selected from the group consisting of Formula (I), Formula (II), Formula (III) or Formula (IV) together with an alpha-adrenergic blocker.

The alpha-adrenergic blocker used in the methods of the invention may, e.g., be selected from a group consisting of terazosin, doxazosin, alfuzosin, and pharmaceutically acceptable salts of any of the foregoing.

In certain embodiments, the compounds are administered in accordance with the formulations and methods of the invention to a patient who is already presenting with symptoms of a neurological disease. In other embodiments, the compounds are administered in accordance with the formulations and methods of the invention to a patient who is already presenting with a cardiovascular disease (e.g., hypertension). In certain embodiments, the compounds are administered in accordance with the formulations and methods of the invention to a patient who is already presenting with symptoms of a neurological disease and a cardiovascular disease (e.g., hypertension). In yet other embodiments, the compounds are administered in accordance with the formulations and methods of the invention to a patient (human subject) who is not presenting with any neurological or cardiovascular problems, but, e.g., may be at risk for future neurological and/or cardiovascular problems.

The antihypertensive drug or an antihypertensive agent used in the methods of the invention may, e.g., include thiazide diuretics, ACE (angiotensin-converting enzyme) inhibitors, and calcium channel blockers (CCBs), Angiotensin II receptor blockers (ARBs), calcium channel blockers (including dihydropyridines and nondihydropyridines) direct vasodilators, diuretics (including potassium-sparing, loop, thiazide, and thiazide-type diuretics), alpha-blockers, and beta-blockers.

The antihypertensive drugs are also considered for purposes of this invention to encompass an alpha-adrenergic blockers, and, in particular terazosin, doxazosin, alfuzosin, and pharmaceutically acceptable salts thereof. In certain embodiments, in addition to blocking alpha-adrenergic receptors, these compounds also bind to and activate adenosine triphosphate (ATP)-producing enzyme in glycolysis. The activation of the adenosine triphosphate (ATP)-producing enzyme in glycolysis may increase energy availability in the brain and may prevent or slow down build-up of neurodegenerative protein(s) in the brain.

The compounds of the invention may be administered either separately or together via a route selected from the group consisting of orally, parenterally, sublingually, via suppository, nasally, topically, transdermally, intravenously, subcutaneously, intraperitoneally and via implant under the skin. In certain embodiments, the compound is administered chronically to treat, prevent, delay, or slow a neurodegenerative disease such as Alzheimer's disease in a mammal, e.g., a human.

In certain preferred embodiments, the co-administered compounds are buntanetap and an alpha-adrenergic blocker, and the buntanetap is administered in an amount as set forth in this disclosure, along with the alpha-adrenergic blocker.

In certain embodiments, the compounds (e.g., buntanetap and an antihypertensive agent) are co-administered to a mammal (e.g., human) who is symptomatic for hypertension. In certain embodiments, the mammal (e.g., human subject or human patient) is demonstrating symptoms of a neurological disorder or a neurodegenerative disease. In certain embodiments, the mammal (e.g., human subject or human patient) is not demonstrating symptoms of a neurological disorder or a neurodegenerative disease.

In certain embodiments, the buntanetap or a pharmaceutically acceptable salt thereof is administered (i) orally in an amount from about 0.01 mg to about 120 mg on a once-a-day basis;

(ii) intravenously in an amount from about 0.01 mg to about 25 mg/day; or (ii) intraperitoneally/intramuscularly (IP/IM) in a dose from about 0.03 to about 70 mg/day.

In certain embodiments, the compound of Formulas (I), (II), and (III), and the antihypertensive (e.g., an alpha-adrenergic blocker) are administered separately but such that they provide overlapping therapeutic effects.

In certain embodiments, the invention is further directed to a pharmaceutical composition, comprising a therapeutically effective amount of a compound selected from the group consisting of Formula (I), Formula (II) and Formula (III):

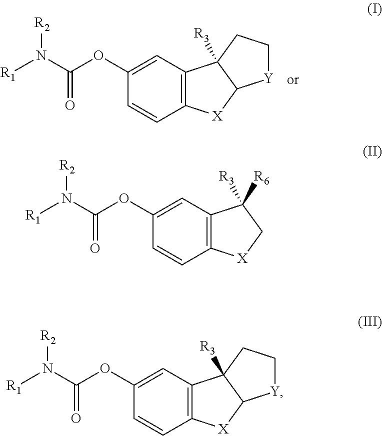

wherein,
in Formula (I) and Formula (II),
   R₁ and R₂ are, independently, hydrogen, branched or straight chain C₁-C₈ alkyl, substituted or unsubstituted aryl, heteroaryl, or aralkyl;
   R₃ is branched or straight chain C₁-C₄ alkyl or heteroalkyl or C₄-C₈ alkyl or heteroalkyl, or substituted or unsubstituted aryl;
   X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
   R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
   R₁ and R₂ are, independently, hydrogen, branched or straight chain C₁-C₈ alkyl, substituted or unsubstituted aryl, heteroaryl, or aralkyl;
   R₃ is branched or straight chain C₁-C₄ alkyl or heteroalkyl or C₄-C₈ alkyl or heteroalkyl, or substituted or unsubstituted aryl;
   X is NR₅, wherein R₅ is C₂₋₈ alkenyl, C₂₋₈ alkynyl, or aralkyl;
   Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof;
   an effective amount of compound(s) which is an antihypertensive agent (e.g., an alpha-adrenergic blocker); and
   at least one pharmaceutically acceptable excipient. In certain preferred embodiments, the compound of Formula (III) is the substantially pure (−)-enantiomer. In certain embodiments, the compound is buntanetap:

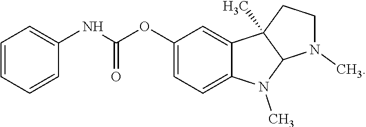 [SMILES: CN1CC[C@]2(C)c3cc(OC(=O)Nc4ccccc4)ccc3N(C)C12]

In certain embodiments, the antihypertensive agent is terazosin or a pharmaceutically acceptable salt thereof and is administered orally once-a-day in an amount that is less than about 0.8 mg, less than about 0.7 mg or less than about 0.6 mg, and buntanetap is administered orally once-a-day in amount from about 2 mg to about 80 mg, about 3 mg to about 60 mg, about 5 mg to about 30 mg, from about 7.5 mg to about 30 mg, from about 10 mg to about 30 mg or from about 15 mg to about 30 mg.

In certain embodiments, the antihypertensive agent is doxazocin or a pharmaceutically acceptable salt thereof and is administered orally once-a-day in an amount that is less than about 0.8 mg, less than about 0.7 mg or less than about 0.6 mg, and buntanetap is administered orally once-a-day in amount from about 2 mg to about 80 mg, about 3 mg to about 60 mg, about 5 mg to about 30 mg, from about 7.5 mg to about 30 mg, from about 10 mg to about 30 mg or from about 15 mg to about 30 mg.

In certain embodiments, the antihypertensive agent is alfuzosin or a pharmaceutically acceptable salt thereof and is administered orally once-a-day in an amount that is less than about 7 mg, less than about 6 mg or less than about 5 mg, and buntanetap is administered orally once-a-day in amount from about 2 mg to about 80 mg, about 3 mg to about 60 mg, about 5 mg to about 30 mg, from about 7.5 mg to about 30 mg, from about 10 mg to about 30 mg or from about 15 mg to about 30 mg.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e. to at least one) of the grammatical objects of the article. By way of example, “an element” means one element or more than one element.

The term “about” will be understood by persons of ordinary skill in the art and will vary to some extent on the context in which it is used. In the context of the present application, the term “about” means a value within 20% (±20%) of the value recited immediately after the term “about,” including the value equal to the upper limit (i.e., ±20%) and the value equal to the lower limit (i.e., −20%) of this range. For example, the phrase “about 100” encompasses any numeric value that is between 80 and 120, including 80 and 120.

The terms “blood pressure medication” and “antihypertensive” and “vasodilator” and “antihypertensive agents” are considered to be synonymous for the purposes of the invention described herein and include drugs capable of lowering blood pressure in a human and agonists and antagonists of the adrenergic receptors.

As used herein, the terms “buntanetap” and “Posiphen” are used interchangeably to refer to (3aR)-1,3a,8-trimethyl-1,2,3,3a,8,8a-hexahydropyrrolo[2,3-b]indol-5-yl phenylcarbamate or a salt thereof.

As used herein, the term “APP” refers to amyloid precursor protein.

As used herein, the term “Aβ” refers to Abeta or amyloid beta or amyloid-β peptide. For purposes of the present invention, these terms are considered to be synonymous.

For purposes of the present invention, a “buntanetap-type” drug or “compounds that are similar to buntanetap as described herein” encompasses Formula (I), Formula (II), Formula (III) or Formula (IV).

As used herein, the term “HTT” refers to huntingtin or the Huntington protein.

As used herein, “TDP43” refers to the TAR-DNA binding protein TDP43.

As used herein, “C9orf72” refers to the C9orf72 protein found in many regions of the brain.

As used herein, the term “neurotoxic aggregating protein” refers to a protein or family of proteins that has neurotoxic effect upon accumulating in a tissue of the brain, such as the brain tissue. Non-limiting examples of neurotoxic aggregating proteins are APP, Aβ, SOD1, SNCA, NAC, TSE amyloid plaque, HTT, Tau, TDP43 and C9orf72.

As used herein, the terms “protein”, “peptide” and “polypeptide” are used interchangeably and refer to a compound comprised of amino acid residues covalently linked by peptide bonds.

By the term “specifically binds,” as used herein, means a molecule, such as an antibody or a small molecule, which recognizes and binds to another molecule or feature, but does not substantially recognize or bind other molecules or features in a sample.

The term “inhibit” as used herein means to reduce a molecule, a reaction, an interaction, a gene, an mRNA, and/or a protein's expression, stability, function, or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function, and activity, e.g., antagonists.

Depending on the disease that is treated, the “therapeutic effect” provided by the methods of the present invention includes, e.g., an improvement in mental function, as determined by a change in a score on a scale for mental function assessment; a reduction in hypertension, a reduction in nocturnal urination, an improvement in sexual function, a reduction in plasma Total Tau level; a decrease in the production of amyloid beta (Aβ); a decrease in the production of tau; a decrease in the production of alpha-Synuclein (αSYN); a decrease in the production of TDP43; and a combination of two or more of any of the foregoing.

In the context of the invention, the “improvement in mental function” may be ascertained by a change in a score on a test known to one of ordinary skill in the art, including e.g., Alzheimer's Disease Assessment Scale-Cognitive Subscale (e.g., “ADAS-Cog 11”, “ADAS-Cog 12”, “ADAS-Cog 13”, “ASAS-Cog 14”), a Mini-Mental State Examination (MMSE), a Montreal Cognitive Assessment (MoCA), Wechsler Intelligence Test, after 3 months of administration, as compared to a score at the start of administration, which is used as a baseline.

In the context of the invention, the improvement in motor function may be ascertained by a test known to one of ordinary skill in the art, including, e.g., by MDS Unified Parkinson's Disease Rating Scale (MDS-UPDRS).

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary, and topical administration.

As used herein, the term “co-administering” refers to a compound of Formula (I), (II), (III) and/or (IV) which is administered to a mammal (e.g., a human subject or human patient) together with an appropriate vasodilator, anti-hypertensive (e.g., an alpha-adrenergic blocker) so that the two classes of agents provide an overlapping effect. While in certain cases it may be possible to administer the two classes of agents in a single dosage form, it is contemplated that these agents may be separately administered either via the same route of administration or different routes of administration to achieve overlapping effects, taking into account their differing physical/chemical properties (including but not limited to solubility, bioavailability, half-life, metabolism, and clearance/elimination from the body, etc.).

“Pharmaceutically acceptable” refers to a material(s) which are compatible with the activity of the compound useful within the invention and which are physiologically acceptable to the patient (e.g., human) from a pharmacological/toxicological point of view and to the manufacturing pharmaceutical chemist from a physical/chemical point of view regarding composition, formulation, stability, patient acceptance, and bioavailability.

“Pharmaceutical acceptable carrier” refers to a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium, and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

An “individual”, “patient”, or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term neurologic disease or disorder is meant to include Alzheimer's disease, tauopathies, Parkinson's and alpha-synucleopathies, Prion's disease, Down Syndrome, Huntington's disease, Amyloid Lateral Sclerosis, Multiple Sclerosis and other dementias and neurodegenerative diseases.

The term “synergy” as used herein means that the amount (i.e., dosage) of each agent alone has no effect and is sub-therapeutic as monotherapy but when administered as a combination is therapeutic.

The term “treat” or “treating” as used herein means reducing the frequency with which symptoms are experienced by a subject or administering the combination of agents or compounds to reduce the frequency and/or severity with which symptoms are experienced. As used herein, “alleviate” is used interchangeably with the term “treat.” Treating a disease, disorder or condition may or may not include complete eradication or elimination of the symptom. Thus, the term “treating” encompasses preventing and slowing a condition described herein.

The term “therapeutic” as used herein means a treatment and/or prophylaxis of a condition or disease state as described herein.

The term “alkyl” as used herein refers to a branched or unbranched saturated hydrocarbon group of 1 to 4, 1 to 8, or 1 to 20 carbon atoms, such as methyl, ethyl, n-propyl, isopropyl, n-butyl, isobutyl, t-butyl, pentyl, hexyl, heptyl, octyl, and the like. Examples of cycloalkyl groups include cyclopentyl and cyclohexyl.

The term “alkenyl” as used herein refers to a hydrocarbon group of 2 to 4, 2 to 8, or 2 to 20 carbon atoms and structural formula containing a carbon-carbon double bond.

The term “alkynyl” as used herein refers to a hydrocarbon group of 2 to 4, 2 to 8, or 2 to 20 carbon atoms and a structural formula containing a carbon-carbon triple bond.

The term “aryl” is defined as any carbon-based aromatic group including, but not limited to, phenyl, benzene, naphthalene, anthracene, phenanthrene, pyrene, and benzo[a]pyrene, etc.

The term “substituted aryl” is defined as an aryl group having at least one group attached to the aryl group that is not hydrogen. Examples of groups that can be attached to the aryl group include, but are not limited to, alkyl, alkynyl, alkenyl, aryl, heterocyclic, halide, nitro, amino, ester, ketone, aldehyde, hydroxy, carboxylic acid, alkoxy, cyano, alkoxy, thioalkyl, haloalkyl, hydroxyalkyl, alkylamino, diakylamino, or acyl. In various embodiments, a substituent is bound to carbon 2, 3, 4, 5, or 6 of one of these moieties. Examples of alkoxy substituents include, but are not limited to, methoxy, ethoxy, and isopropoxy groups. Examples of acyl substituents include acetyl and benzoyl groups.

The term “aralkyl” is defined as an aryl group having an alkyl, alkynyl, or alkenyl group attached to the aryl group. An example of an aralkyl group is a benzyl group.

The term “heteroaryl” is defined as an aryl group that has at least one heteroatom such as nitrogen, sulfur, or oxygen incorporated within the ring of the aryl group.

The term “heteroalkyl” is defined as an alkyl group that has at least one heteroatom, such as nitrogen, sulfur, oxygen, or phosphate, incorporated within the alkyl group or attached to the alkyl group.

### BRIEF DESCRIPTION OF THE DRAWINGS

FIG. 1 is a Western blot and a graph showing that buntanetap lowers APP in vitro in a dose-dependent manner in SH-SY-5Y human neuroblastoma cells.

FIG. 2A is a Table, collection of graphs and a Western blot showing that buntanetap treatment of APP/PS1 transgenic AD mice reduced APP and its fragments in hippocampus.

FIG. 2A is a Table, collection of graphs and a Western blot showing that buntanetap treatment of APP/PS1 transgenic AD mice reduced APP and its fragments in hippocampus.

FIG. 2B shows dose-dependent improvement in cognition in the population with confirmed early AD.

FIG. 2C shows that Buntanetap's efficacy is strongly correlated with MMSE status.

FIG. 3 is a table showing the reduction of APP/Abeta, tau/phospho-tau and alpha-synuclein in the spinal fluid of mildly cognitive impaired patients.

### DETAILED DESCRIPTION OF THE INVENTION

The invention is directed in part to a method to inhibit, prevent or treat a neurodegenerative disease such as, e.g., Alzheimer's disease, dementia with Lewy bodies, frontotemporal dementia, other dementias, chronic traumatic encephalopathy, tauopathies, Parkinson's disease, alpha-synucleopathies, Prion's disease, Down Syndrome, Huntington's disease, Amyloid Lateral Sclerosis, multiple sclerosis, and other, neurodegenerative diseases which present as misfolding, aggregation and accumulation of proteins in the brain, resulting in inflammation and eventual cell death; and hypertension via the administration of buntanetap, compounds that are similar to buntanetap as described herein (e.g., a drug encompassing Formula (I), Formula (II), Formula (III) or Formula (IV)), pharmaceutically acceptable salts and complexes thereof, together with an anti-hypertensive agent (e.g. an alpha-adrenergic blocker), and one or more pharmaceutically acceptable excipients.

Buntanetap is a selective inhibitor of amyloid precursor protein (APP) production and has potential utility as a disease modifying treatment for AD (Cullen 2006; Utsuki 2006; Lahiri 2007). Buntanetap was discovered at the National Institute on Aging and was selected from a series of structurally related compounds designed for APP specificity with no or minimal acetylcholinesterase inhibitory activity. Buntanetap was shown to reduce APP and consequently beta-amyloid (Aβ) production in relevant preclinical in vitro and in vivo studies. Maccecchini, et al., “Buntanetap as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902, hereby incorporated by reference, reported the results of a study of buntanetap single and multiple ascending dose phase 1 randomized, double blind, placebo-controlled safety, tolerance, pharmacokinetic studies were undertaken in 120 healthy human volunteers to define a dose that was then used in a small non-randomized study of five MCI subjects. Buntanetap doses up to 4×60 mg daily X 10 days were well tolerated. In plasma buntanetap, at all doses, was absorbed rapidly (Tmax=1.2 to 1.7 h) and cleared from the circulation biphasically (terminal half-life of 4.3-4.7 h). Buntanetap proved well tolerated and significantly lowered CSF levels of sAPPα, sAPPβ, t-tau, p-tau, and specific inflammatory markers, and demonstrated a trend to lower CSF Aβ₄₂. Buntanetap's activity is also described in Applicant's U.S. Pat. No. 10,383,851, hereby incorporated by reference. Phase II data for Buntanetap has been published, C. Fang et al, Buntanetap, a Novel Translational Inhibitor of Multiple Neurotoxic Proteins, Proves to Be Safe and Promising in Both Alzheimer's and Parkinson's Patients, J Prev Alzheimers Dis (2022). https://doi.org/10.14283/jpad.2022.84, published 10 Oct. 2022, hereby incorporated by reference in its entirety. This publication reported the results of a Phase 2a Clinical Study which was a double-blind, placebo-controlled, multi-center study of 14 early AD patients and 54 early PD patients. AD patients were given either 80 mg buntanetap or placebo QD. PD patients were given 5 mg, 10 mg, 20 mg, 40 mg, 80 mg buntanetap or placebo QD. The primary endpoint was safety and tolerability; secondary endpoint is pharmacokinetics of buntanetap in plasma. The buntanetap was well tolerated at safe at doses up to 80 mg in both AD and PD patients. Cmax and AUC increased with dose without evidence for a plateau up to 80 mg QD. Biomarker data indicated a trend in lowering levels of neurotoxic proteins and inflammatory factors and improving axonal integrity and synaptic function in both AD and PD cohorts. Psychometric tests showed statistically significant improvements in ADAS-Cog11 and WAIS coding in AD patients and MDS-UPDRS and WAIS coding in PD patients.

Buntanetap®, developed by QR Pharma, Inc. (now Annovis Bio, Inc.), is a small molecule that lowers soluble APP protein levels through a post-transcriptional mechanism. Buntanetap is also known as (+)-phenserine. Buntanetap is the stereoisomer of (−)-phenserine (−)-N-phenylcarbamoyl eseroline), which reached clinical assessment for AD as an anticholinesterase inhibitor. Phenserine is an AChE inhibitor which was investigated as being suitable as an agent for therapy for cognitive impairments associated with aging and Alzheimer's disease (U.S. Pat. No. 5,409,948). Due to its high cholinomimetic side effects, phenserine failed in 3 phase 3 clinical studies.

As used herein, the term “buntanetap” refers to (3aR)-1,3a,8-trimethyl-1,2,3,3a,8,8a-hexahydropyrrolo[2,3-b]indol-5-yl phenylcarbamate, with the chemical structure shown in Formula IV below, at a chemical purity of at least 90%, preferably at least 95%, at least 98%, at least 99%, at least 99.5%, at least 99.9% or 100%, having the chemical structural as follows:

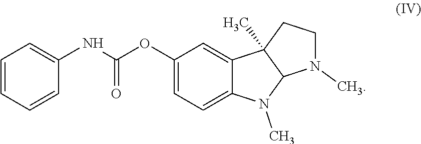 [SMILES: CN1CC[C@]2(C)c3cc(OC(=O)Nc4ccccc4)ccc3N(C)C12]

The term “chemical purity” as applied to (3aR)-1,3a,8-trimethyl-1,2,3,3a,8,8a-hexahydropyrrolo[2,3-b]indol-5-yl phenylcarbamate or a pharmaceutically acceptable salt of buntanetap means the percent by weight of (3aR)-1,3a,8-trimethyl-1,2,3,3a,8,8a-hexahydropyrrolo[2,3-b]indol-5-yl phenylcarbamate or the pharmaceutically acceptable salt of buntanetap in terms of (3aR)-1,3a,8-trimethyl-1,2,3,3a,8,8a-hexahydropyrrolo[2,3-b]indol-5-yl phenylcarbamate or the pharmaceutically acceptable salt of buntanetap and other chemical impurities, e.g., its (−)-enantiomer, that may be present.

The invention also encompasses active metabolites of buntanetap. Active metabolites have previously been identified and include, for example, “N¹-nor-Buntanetap” (which refers to (3aR)-3a,8-dimethyl-1,2,3,3a,8,8a-hexahydropyrrolo[2,3-b]indol-5-yl phenylcarbamate) or a salt thereof; “N⁸-nor-Buntanetap” (which refers to (3aR)-1,3a-dimethyl-1,2,3,3a,8,8a-hexahydropyrrolo[2,3-b] indol-5-yl phenylcarbamate) or a salt thereof; and “N¹,N⁸-nor-Buntanetap” (which refers to (3aR)-3a-methyl-1,2,3,3a,8,8a-hexahydropyrrolo[2,3-b]indol-5-yl phenylcarbamate) or a salt thereof.

In other embodiments, the methods of the present invention are practiced using a phenserine or phenserine-like compound, metabolite, enantiomer, or derivative thereof, known to those skilled in the art, such as those described in U.S. Pat. Nos. 5,171,750; 6,410,747; 6,683,105, 7,153,882; 7,786,162; 7,973,057; 8,258,172; 8,546,430; 8,691,864; and 8,853,253, all of which are incorporated by reference in their entireties.

In a broader sense, the invention is directed in part to the coadministration of a buntanetap-type compound as defined by Formula (I), Formula (II), Formula (III) or Formula (IV) together with an antihypertensive agent.

The buntanetap-type compounds include compounds having the Formula I or II as follows:

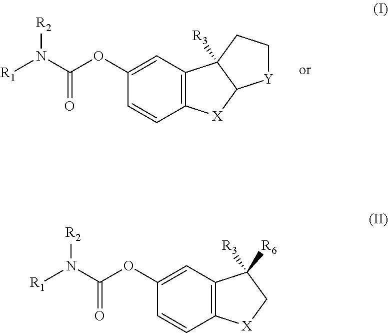

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4, along with an effective amount of an antihypertensive agent.

The chiral center of compounds of Formula I and II is the carbon atom that has R₃ bonded to it. As depicted herein, the (+)-enantiomer has R₃ pointing behind the plane of the page. Although only the (+)-isomer is illustrated to save space, in other embodiments the compound having the Formula I or II can be the (+)-isomer, (−)-isomer, and mixtures of both isomers (e.g., racemic mixtures, including 1:1 racemic mixtures) of all of the compounds encompassed by the invention.

In certain embodiments, the compounds having the Formula I or II have an enantiomeric purity for the (+)-enantiomer of from 55 to 100%, desirably from 75 to 100%, more desirably from 85 to 100%, more desirably from 95 to 100%, and even more desirably 100%.

In certain preferred embodiments, wherein the compound having the Formula I or II is the substantially pure (+)-enantiomer.

In one embodiment, when the compound is Formula I, R₃ is methyl and X is NCH₃.

In one embodiment, when the compound is Formula I or II, R₃ is not methyl. In particular embodiments, R₃ is a branched or straight chain alkyl or heteroalkyl group of 2, 3, 4, 5, 6, 7, or 8 carbons or substituted or unsubstituted aryl.

In another embodiment, when the compound is Formula I or II, Y is C(H)R₄ or X is O, S, or C(H)R₄.

In another embodiment, when the compound is Formula I, R₃ is methyl, X is NCH₃, and Y is NCH₃. In one embodiment, when the compound is Formula I, R₃ is methyl, X is NCH₃, Y is NCH₃, and R₁ is C₁-C₈ straight chain alkyl or benzyl and R₂ is hydrogen. In one embodiment, when the compound is Formula I, R₃ is methyl, X is NCH₃, Y is NCH₃, and R₁ is substituted or unsubstituted phenyl and R₂ is hydrogen. In one embodiment, when the compound is Formula I, R₃ is methyl, X is NCH₃, Y is NCH₃, and R₁ and R₂ are, independently, methyl or ethyl.

In another embodiment, when the compound is Formula I, R₃ is methyl, X is NCH₃, and Y is O. In one embodiment, when the compound is Formula I, R₃ is methyl, X is NCH₃, Y is O, R₁ is C₁-C₈ straight chain alkyl or benzyl, and R₂ is hydrogen. In one embodiment, when the compound is Formula I, R₃ is methyl, X is NCH₃, Y is O, and R₁ and R₂ are, independently, methyl or ethyl. In one embodiment, when the compound is Formula I, R₃ is methyl, X is NCH₃, Y is O, and R₁ is substituted or unsubstituted phenyl and R₂ is hydrogen.

In another embodiment, when the compound is Formula I, R₃ is methyl, X is NCH₃, and Y is S. In one embodiment, when the compound is Formula I, R₃ is methyl, X is NCH₃, Y is S, R₁ is C₁-C₈ straight chain alkyl or benzyl, and R₂ is hydrogen. In one embodiment, when the compound is Formula I, R₃ is methyl, X is NCH₃, Y is S, and R₁ and R₂ are, independently, methyl or ethyl. In one embodiment, when the compound is Formula I, R₃ is methyl, X is NCH₃, Y is S, R₁ is substituted or unsubstituted phenyl, and R₂ is hydrogen.

In another embodiment, when the compound is Formula I, R₃ is methyl, X is NCH₃, and Y is NR₅. In one embodiment, when the compound is Formula I, R₃ is methyl, X is NCH₃, and Y is NR₅, wherein R₅ is —CH₂CH═CH₂, —CH₂CH₂Ph, benzyl, or hydrogen.

In another embodiment, when the compound has the Formula I, R₃ is methyl, Y is NCH₃, and X is NCH₃, wherein R₄ is benzyl or hydrogen.

In another embodiment, when the compound is Formula I, R₃ is methyl, X is NCH₃, Y is NR₅, wherein each R₄ and R₅ is, independently, hydrogen or benzyl.

In another embodiment, when the compound is Formula I, R₃ is phenyl, X is NCH₃, and Y is NCH₃.

In another embodiment, when the compound is Formula I, R₃ is methyl, and X is NCH₃, and Y is not NH or NHCH₂Ph.

In some embodiments, when the compound is Formula I, R₁ and R₂ are independently, hydrogen, substituted or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ and R₂ are independently, hydrogen or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ is hydrogen, R₂ is unsubstituted aryl, R₃ is methyl, X and Y are independently NR₅, wherein R₅ is independently hydrogen or methyl.

In a certain preferred embodiment, when the compound is Formula I, R₁ is hydrogen, R₂ is phenyl, R₃ is methyl, X is NCH₃, and Y is NCH₃.

In other embodiment, when the compound is Formula I, R₁ is hydrogen, R₂ is phenyl, R₃ is methyl, X is NCH₃, and Y is NH.

In a certain preferred embodiment, when the compound is Formula I, R₁ is hydrogen, R₂ is phenyl, R₃ is methyl, X is NH, and Y is NCH₃.

In a certain preferred embodiment, when the compound is Formula I, R₁ is hydrogen, R₂ is phenyl, R₃ is methyl, X is NH, and Y is NH.

In another embodiment, when the compound is Formula II, R₃ is methyl, X is C (H) CH₃, and R₆ is (CH₂)₂R₇, where R₇ is a substituted or unsubstituted amino group.

In a certain preferred embodiment, when the compound is Formula II, R₃ is methyl, X is NCH₃, and R₆ is (CH₂)₂R₇, where R₇ is a substituted or unsubstituted amino group.

In a certain preferred embodiment, wherein the compound having the Formula II is the substantially pure (+)-enantiomer.

The invention also relates to the use of a compound having the Formula (III) as follows:

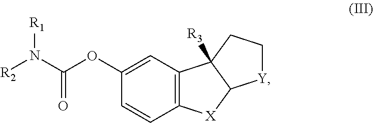

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl.

As depicted herein, the (−)-enantiomer has R₃ pointing in front of the plane of the page. Although only the (−)-isomer is illustrated to save space, in other embodiments the compound having the Formula (III) can be the (+)-isomer, (−)-isomer, and mixtures of both isomers (e.g., racemic mixtures, including 1:1 racemic mixtures) of all of the compounds encompassed by the invention.

In a certain preferred embodiment, wherein the compound having the Formula (III) is the substantially pure (−)-enantiomer.

In one embodiment, when the compound is Formula (III), X is NR₅, wherein R₅ is aralkyl.

In one embodiment, when the compound is Formula (III), X and Y are NR₅, wherein R₅ is aralkyl.

In one embodiment, when the compound is Formula (III), wherein X is NR₅, wherein R₅ is aralkyl, and Y is NR₅, wherein R₅ is branched or straight chain C₁₋₈ alkyl or heteroalkyl.

In one embodiment, when the compound is Formula (III), wherein R₁ is branched or straight chain C₁-C₈alkyl, aralkyl or aryl, R₂ is hydrogen, branched or straight chain C₁-C₈ alkyl, substituted or unsubstituted aryl, or aralkyl; Y is NR₅, wherein R₅ is aralkyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NR₅ where R₅ is benzyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈alkyl, aralkyl or aryl; R₂ is hydrogen, branched or straight chain C₁-C₈ alkyl; Y is NR₅ where R₅ is benzyl; and X is NR₅, wherein R₅ is hydrogen.

In one embodiment, when the compound is Formula (III), wherein R₁ is para-halophenyl; Y is NCH₃; and X is NR₅, wherein R₅ is alkyl or aralkyl, wherein R₁ is not para-phenyl bromophenyl when R₅ is benzyl.

In one embodiment, when the compound is Formula (III), wherein R₁ is para-isopropyl phenyl; R₂ is hydrogen; R₃ is methyl; Y is NR₅ where R₅ is benzyl; and X is NR₅, wherein R₅ is hydrogen.

Encompassed in the formulations of the invention are the (+)-isomer, (−)-isomer, and mixtures of both isomers (e.g., racemic 1:1 mixtures) of all of the compounds of the invention unless such compounds are specifically excluded.

Variables, such as R₁-R₇, n, X and Y throughout the application are the same variables as previously defined unless stated to the contrary.

The compounds described herein may form salts with acids or bases, and such salts are included in the present invention. In one embodiment, the salts are pharmaceutically acceptable salts. The term “salts” embraces addition salts of free acids or free bases that are compounds of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification, or formulation of compounds of the invention.

Examples of the pharmaceutically acceptable salt of buntanetap include acid addition salts prepared from a suitable acid. The suitable acid can be hydrobromic acid, hydrochloric acid, hydroiodic acid, sulfuric acid, carbonic acid, nitric acid, phosphoric acid, tetrafluoroboronic acid, perchloric acid, 1-hydroxy-2-naphthoic acid, 2,2-dichloroacetic acid, methanesulfonic acid, trifluoromethanesulfonic acid, 2-hydroxyethanesulfonic acid, benzenesulfonic acid, p-toluenesulfonic acid, cyclohexylaminosulfonic acid, 2-oxoglutaric acid, 4-acetamidobenzoic acid, acetic acid, phenylacetic acid, propionic acid, formic acid, succinic acid, glycolic acid, gluconic acid, malic acid, lactic acid, tartaric acid, citric acid, glucuronic acid, ascorbic acid, maleic acid, fumaric acid, pyruvic acid, aspartic acid, glutamic acid, benzoic acid, 4-hydroxybenzoic acid, anthranilic acid, 4-hydroxybenzoic acid, mandelic acid, pamoic acid, pantothenic acid, sulfanilic acid, stearic acid, alginic acid, β-hydroxybutyric acid, salicylic acid, galactaric acid and galacturonic acid. Preferably, the pharmaceutically acceptable salt is buntanetap tartrate, i.e., the acid addition salt of tartaric acid.

Suitable pharmaceutically acceptable base addition salts of compounds of the invention include, for example, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N methylglucamine) and procaine. Examples of pharmaceutically unacceptable base addition salts include lithium salts and cyanate salts. All of these salts may be prepared from the corresponding compound by reacting, for example, the appropriate acid or base.

In certain preferred embodiments, the (buntanetap) compound(s) of the present invention is coadministered with an antihypertensive drug or a vasodilator. Part of the physiological process of vasodilation involves the release of nitric oxide by vascular endothelial cells which then diffuses to nearby vascular smooth muscle cells. There, nitric oxide activates soluble guanylate cyclase which converts guanosine triphosphate (GTP) to cyclic guanosine monophosphate (cGMP), the main effector of the system.

Hypertension is one of the leading modifiable risk factors for Alzheimer disease and related dementias (ADRD). See, e.g., Yasar, S. et al. “Antihypertensive Drugs Decrease Risk of Alzheimer Disease, Neurology. 2013 Sep. 3; 81 (10): 896-903. doi: 10.1212/WNL.0b013e3182a35228; Livingston G, Huntley J, Sommerlad A, et al. Dementia prevention, intervention, and care: 2020 report of the Lancet Commission. Lancet. 2020; 396 (10248): 413-446. doi: 10.1016/S0140-6736 (20) 30367-6; National Academies of Sciences, Engineering, and Medicine. Preventing Cognitive Decline and Dementia: A Way Forward. National Academies Press; 2017. Kehoe P G. The coming of age of the angiotensin hypothesis in Alzheimer's disease: progress toward disease prevention and treatment? J Alzheimers Dis. 2018; 62 (3): 1443-1466. doi: 10.3233/JAD-171119. Mechanistic, observational, and randomized clinical studies suggest that certain classes of antihypertensive medication may reduce cognitive impairment independent of their blood pressure-lowering effects. Kehoe P G. The coming of age of the angiotensin hypothesis in Alzheimer's disease: progress toward disease prevention and treatment? J Alzheimers Dis. 2018; 62 (3): 1443-1466. doi: 10.3233/JAD-171119; Levi Marpillat N, Macquin-Mavier I, Tropeano A-I, Bachoud-Levi A-C, Maison P. Antihypertensive classes, cognitive decline, and incidence of dementia: a network meta-analysis. J Hypertens. 2013; 31 (6): 1073-1082 doi: 10.1097/HJH.0b013e3283603f53; den Brok MGHE, van Dalen J W, Abdulrahman H, et al. Antihypertensive medication classes and the risk of dementia: a systematic review and network meta-analysis. J Am Med Dir Assoc. 2021; 22 (7): 1386-1395. doi: 10.1016/j.jamda.2020.12.019; Hajjar I, Okafor M, McDaniel D, et al. Effects of candesartan vs lisinopril on neurocognitive function in older adults with executive mild cognitive impairment: a randomized clinical trial. JAMA Netw Open. 2020; 3 (8): e2012252. doi: 10.1001/jamanetworkopen.2020.12252; ClinicalTrials.gov. Risk Reduction for Alzheimer's Disease (rrAD). ClinicalTrials.gov identifier: NCT02913664. Accessed Jun. 19, 2022. https://clinicaltrials.gov/ct2/show/NCT02913664.

Previous observational studies have reported that prevalent use of regimens containing antihypertensive medications that stimulate type 2 and 4 angiotensin II receptors, compared with those that do not, were associated with lower rates of incident mild cognitive impairment and dementia. “Association of New Use of Antihypertensives That Stimulate vs Inhibit Type 2 and 4 Angiotensin II Receptors With Dementia Among Medicare Beneficiaries”, Zachary A. Marcum, PharmD, PhD; Nico Gabriel, M A; Adam P. Bress, PharmD, M S; Inmaculada Hernandez, PharmD, PhD open access JAMA Netw Open. 2023; 6 (1): e2249370. doi: 10.1001/jamanetworkopen.2022.49370. However, antihypertensive agents are generally deprescribed in late-stage dementia, according to the Medican Appropriates Tool for Comorbid Health Conditions in Dementia (Match-D) Page A T, Potter K, Clifford R, McLachlan A J, Etherton-Beer C (October 2016). “Medication appropriateness tool for co-morbid health conditions in dementia: consensus recommendations from a multidisciplinary expert panel”. Internal Medicine Journal. 46 (10): 1189-1197. doi: 10.1111/imj.13215. PMC 5129475. PMID 27527376. Further, as reported by Ding, J. et al “Antihypertensive Medications and Risk for Incident Dementia and Alzheimer's Disease: a Meta-Analysis of Individual Participant Data From Prospective Cohort Studies” The Lancet Neurology, Volume 19, Issue 1, p 61-70, January 2020, over a long period of observation, no evidence was found that a specific antihypertension medication drug class was more effective than others in lowering risk of dementia. Among people with hypertensive levels of blood pressure, use of any such medication was suggested as possibly reducing the risk for dementia.

There are at least six major classes of antihypertensive drugs-diuretics, β-blockers, alpha-blockers, calcium channel blockers, renin-angiotensin system inhibitors (including angiotensin-converting enzyme inhibitors), and angiotensin receptor blockers. Antihypertensive agents include but are not limited to, e.g., thiazide diuretics, ACE (angiotensin-converting enzyme) inhibitors, and calcium channel blockers. Angiotensin II receptor blockers (ARBs) and calcium channel blockers (CCBs) (including dihydropyridines and nondihydropyridines), vasodilators, diuretics (including potassium-sparing, loop, thiazide, and thiazide-type diuretics), alpha blockers and beta-blockers.

Calcium channel blockers include but are not limited to dihydropyridines such as amlodipine barnidipine, cilnidipine, clevidipine, felodipine, isradipine, lercanidipine, levamlodipine, nicardipine, nifedipine, nimodipine, nisoldipine and nitrendipine. The calcium channel blockers that non-dihyopyridines such as diltiazem and verapamil.

ACE inhibitors inhibit the activity of angiotensin-converting enzyme (ACE), an enzyme responsible for the conversion of angiotensin I into angiotensin II, a potent vasoconstrictor. They include but are not limited to captopril, enalapril, fosinopril, lisinopril, moexipril, perindopril, quinapril, ramipril, trandolapril and benazepril.

Angiotensin II receptor antagonists (ARBs) work by antagonizing the activation of angiotensin receptors. These agents include but are limited to azilsartan, candesartan, eprosartan, irbesartan, losartan, olmesartan, telmisartan, valsartan and fimasartan.

Adrenergic receptor antagonists include but are not limited to alpha blockers and beta blockers.

Beta blockers includes but are not limited to acebutolol, atenolol, bisoprolol, betaxolol, carteolol, carvedilol, labetalol, metoprolol, nadolol, nebivolol, oxprenolol, penbutolol, pindolol, propranolol and timolol.

Vasodilators act directly on the smooth muscle of arteries to relax their walls so blood can move more easily through them; they are only used in hypertensive emergencies or when other drugs have failed, and even so are rarely given alone. Examples of vasodilators include sodium nitroprusside, a very potent, short-acting vasodilator, hydralazine and minoxidil.

Alpha blockers include but are not limited to doxazosin, terazosin, chlorpromazine, phentolamine, indoramin, phenoxybenzamine, prazosin, terazosin, tolazoline and urapidil.

Mixed Alpha and Beta blockers include but are not limited to bucindolol, carvedilol, labetalol, and clonidine.

Antihypertensive agents further include renin inhibitors such as aliskiren and indirect anti-adrenergics including guanethidine, mecamplamine and reserpine.

Diuretics facilitate diuresis and include, e.g., hydrochlorothiazide and furosemide. Classes of diuretics include thiazides, loop, potassium sparing/aldosterone antagonists, vasodilators, centrally acting agents, and ganglion blockers.

Thiazide diuretics include but are not limited to epitizide, hydrochlorothiazide and chlorothiazide, bendroflumethiazide, methyclothiazide and polythiazide. Thiazide-like diuretics include but are not limited to indapamide, chlorthalidone, metalozone, xipamide and clopamide.

Loop diuretics include but are not limited to bumetanide, ethacrynic acid, furosemide, torsemide.

Potassium-sparing diuretics include but are not limited to amiloride, triamterene, spironolactone and eplerenone.

Centrally acting agents include clonidine, methyldopa.

Ganglion blockers include trimetaphan.

Antihypertensives also include aldosterone receptor antagonists such as eplerenone, and spironolactone; alpha-2 adreneric receptor agonists such as clonidine, guanabenz, guanfacine, methyldopa and moxonidine; ganglion blocking agents such as trimetaphan, potassium sparing aldosterone antagonists such as amiloride and spironolactone and endothelium receptor blockers which include but are not limited to bosentan (which work by blocking endothelin receptors).

### Administration and Dosing

In the methods of the invention, buntanetap, its analogs, metabolites, or a pharmaceutically acceptable salt thereof can be administered parenterally, enterally or transdermally. Examples of the route of administration of buntanetap, or an analog, metabolite, or pharmaceutically acceptable salt or similar compound thereof are intravenous, intraocular, intramuscular, subcutaneous, topical, oral, sublingual, and buccal. Preferably, for purposes of the present invention, buntanetap is administered orally.

In the present invention, buntanetap, or a pharmaceutically acceptable salt of buntanetap, can be administered once, twice, three times, or four times daily. In certain embodiments, buntanetap is preferably administered on a once-a-day basis. Depending on the route of administration, buntanetap is administered in different dose ranges.

In certain embodiments, buntanetap, or a pharmaceutically acceptable salt of buntanetap, is administered orally in an amount from about 0.01 mg to about 120 mg, preferably on a once-a-day basis. In certain preferred embodiments, buntanetap, or a pharmaceutically acceptable salt of buntanetap, is administered in an amount from about 0.01 mg to about 10 mg, from about 0.01 mg to about 5 mg, or from about 0.01 mg to about 0.9 mg, preferably on a once-a-day basis.

In certain embodiments, the compounds are administered in accordance with the formulations and methods of the invention to a patient who is already presenting with symptoms of a neurological disease. In other embodiments, the compounds are administered in accordance with the formulations and methods of the invention to a patient who is already presenting with hypertension. In yet other embodiments, the compounds are administered in accordance with the formulations and methods of the invention to a patient (human subject) who is not presenting with any neurological or hypertensive problems.

In certain embodiments of each of the methods of the present invention as described above, the oral pharmaceutical composition includes from about 0.01 mg to about 120 mg buntanetap or a pharmaceutically acceptable salt thereof, the IP/IM pharmaceutical composition includes from about 0.03 to about 70 mg buntanetap or a pharmaceutically acceptable salt thereof, and the intravenous (IV) pharmaceutical formulation includes from about 0.1 to about 25 mg buntanetap or a pharmaceutically acceptable salt thereof.

In general, the dose of buntanetap preferred to be administered to healthy human patients is a tolerable dose, i.e., a dose that does not cause untoward side effects in a majority of human patient, which dose is also effective for prophylactic treatment of the healthy human(s) with respect to, e.g., neurodegenerative diseases, cancer, cardiovascular homeostasis, diseases or conditions of vital organs, cardiovascular disease, and the like.

In certain embodiments of the methods described herein, peak plasma circulating levels of buntanetap in humans range, e.g., from about 0.1 ng/mL to about 40 ng/mL, in certain embodiments from about 0.2 ng/mL to about 2 ng/ml, and more preferably from about 0.3 ng/mL to about 12 ng/mL. In certain preferred embodiments, the peak plasma circulating level is reached within about 6 hours after administration of buntanetap to humans. In certain embodiments, the peak plasma circulating level is reached within about 3 hours after administration of buntanetap to the humans. In certain embodiments, the plasma circulating level of buntanetap is equal to or greater than about 0.01 ng/ml, 0.05 ng/mL, 0.1 ng/mL, 0.2 ng/ml, 3 ng/ml, 4 ng/ml, 5 ng/ml, 6 ng/ml, 7 ng/ml, 8 ng/mL, 9 ng/mL, 10 ng/mL, 11 ng/ml, 12 ng/ml, 13 ng/ml, 14 mg/mL, 15 ng/ml, 16 ng/ml, 17 ng/mL, 18 ng/mL, 19 ng/mL, or 20 ng/mL for at least 9 hours, and preferably for at least 12 hours, after administration of buntanetap to humans. In certain embodiments, the half-life of buntanetap in cerebrospinal fluid after administering is about 12 hours, and the half-life of buntanetap in plasma after administering is about 5 hours.

The therapeutic agent(s) used as the buntanetap-type drug and antihypertensive drug in the formulations and treatments of the present invention are preferably dosed in therapeutically effective amounts known to those skilled in the art. In certain embodiments, the therapeutically effective amount is an amount that yields a maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. In other embodiments, the therapeutic amount of the antihypertensive drug (e.g., an alpha-adrenergic blocker) is subtherapeutic (meaning that the dose of the antihypertensive drug is lower than the lowest amount approved (e.g., by a governmental regulatory agency such as the U.S. FDA) for treating hypertension or a lower dose as compared to that drug being administered without the buntanetap-type drug). One skilled in the clinical and pharmacological arts will be able to determine a therapeutically effective amount through routine experimentation, namely by monitoring a subject's response to administration of the agent and adjusting the dosage accordingly. In certain embodiments, the antihypertensive agent is administered to the patient at appropriate time intervals (e.g., concurrently with the buntanetap) and via an appropriate route of administration (e.g., oral, subcutaneous, intravenous, intramuscular). In certain embodiments, the antihypertensive drug (e.g., an alpha-adrenergic blocker) is administered together with the buntanetap-type drug in a single formulation where possible.

The antihypertensive drug(s) may be administered in a therapeutic dose, examples of which are set forth below:

| | | Usual Dose | |
|---|---|---|---|
| | | Range in | Usual Daily |
| Class | Drug (Trade Name) | mg/Day | Frequency* |
| Thiazide diuretics | chlorothiazide (Diuril ®) | 125-500 | 1-2 |
| | chlorthalidone (generic) | 12.5-25 | 1 |
| | hydrochlorothiazide | 12.5-50 | 1 |
| | (Microzide, HydroDIURIL ®) | | |
| | polythiazide (Renese ®) | 2-4 | 1 |
| | indapamide (Lozol ®) | 1.25-2.5 | 1 |
| | metolazone (Mykrox ®) | 0.5-1.0 | 1 |
| | metolazone (Zaroxolyn ®) | 2.5-5 | 1 |
| Loop diuretics | bumetanide (Bumex ®) | 0.5-2 | 2 |
| | furosemide (Lasix ®) | 20-80 | 2 |
| | torsemide (Demadex ®) | 2.5-10 | 1 |
| Potassium-sparing diuretics | amiloride (Midamor ®) | 5-10 | 1-2 |
| | triamterene (Dyrenium ®) | 50-100 | 1-2 |
| Aldosterone receptor | eplerenone (Inspra ®) | 50-100 | 1 |
| blockers | | | |
| | spironolactone (Aldactone ®) | 25-50 | 1 |
| Beta blockers | atenolol (Tenormin ®) | 25-100 | 1 |
| | betaxolol (Kerlone ®) | 5-20 | 1 |
| | bisoprolol (Zebeta ®) | 2.5-10 | 1 |
| | metoprolol (Lopressor ®) | 50-100 | 1-2 |
| | metoprolol extended release | 50-100 | 1 |
| | (Toprol XL ®) | | |
| | nadolol (Corgard ®) | 40-120 | 1 |
| | propranolol (Inderal ®) | 40-160 | 2 |
| | propranolol long-acting | 60-180 | 1 |
| | (Inderal LA ®) | | |
| | timolol (Blocadren ®) | 20-40 | 2 |
| Beta blockers with intrinsic | acebutolol (Sectral ®) | 200-800 | 2 |
| sympathomimetic activity | | | |
| | penbutolol (Levatol ®) | 10-40 | 1 |
| | pindolol (generic) | 10-40 | 2 |
| Combined alpha- and Beta | carvedilol (Coreg ®) | 12.5-50 | 2 |
| blockers | | | |
| | labetalol (Normodyne, | 200-800 | 2 |
| | Trandate ®) | | |
| ACE Inhibitors | benazepril (Lotensin ®) | 10-40 | 1 |
| | captopril (Capoten ®) | 25-100 | 2 |
| | enalapril (Vasotec ®) | 5-40 | 1-2 |
| | fosinopril (Monopril ®) | 10-40 | 1 |
| | lisinopril (Prinivil ®, Zestril ®) | 10-40 | 1 |
| | moexipril (Univasc ®) | 7.5-30 | 1 |
| | perindopril (Aceon ®) | 4-8 | 1 |
| | quinapril (Accupril ®) | 10-80 | 1 |
| | ramipril (Altace ®) | 2.5-20 | 1 |
| | trandolapril (Mavik ®) | 1-4 | 1 |
| Angiotensin II antagonists | candesartan (Atacand ®) | 8-32 | 1 |
| | eprosartan (Teveten ®) | 400-800 | 1-2 |
| | irbesartan (Avapro ®) | 150-300 | 1 |
| | losartan (Cozaar ®) | 25-100 | 1-2 |
| | olmesartan (Benicar ®) | 20-40 | 1 |
| | telmisartan (Micardis ®) | 20-80 | 1 |
| | valsartan (Diovan ®) | 80-320 | 1-2 |
| | Azilsartan medoxomil | 40-80 | 1 |
| | (Edarbi ®) | | |
| Calcium Channel blockers- | diltiazem extended release | 180-420 | 1 |
| nondihydropyridines | (Cardizem CD ®, Dilacor | | |
| | XR ®, Tiazac ®) | | |
| | diltiazem extended release | 120-540 | 1 |
| | (Cardizem LA ®) | | |
| | verapamil immediate release | 80-320 | 2 |
| | (Calan ®, Isoptin ®) | | |
| | verapamil long acting (Calan | 120-480 | 1-2 |
| | SR ®, Isoptin SR ®) | | |
| | verapamil (Coer ®, Covera | 120-360 | 1 |
| | HS ®, Verelan PM ®) | | |
| Calcium channel blockers- | amlodipine (Norvasc ®) | 2.5-10 | 1 |
| dihydropyridines | | | |
| | felodipine (Plendil ®) | 2.5-20 | 1 |
| | isradipine (Dynacirc ® CR) | 2.5-10 | 2 |
| | nicardipine sustained release | 60-120 | 2 |
| | (Cardene SR ®) | | |
| | nifedipine long-acting (Adalat | 30-60 | 1 |
| | CC ®, Procardia XL ®) | | |
| | nisoldipine (Sular ®) | 10-40 | 1 |
| Alpha-1 blockers | doxazosin (Cardura ®) | 1-16 | 1 |
| | prazosin (Minipress ®) | 2-20 | 2-3 |
| | terazosin (Hytrin ®) | 1-20 | 1-2 |
| | alfuzosin (Uroxatral ®) | 7.5-40 | 1 |
| Central alpha-2 agonists and | clonidine (Catapres ®) | 0.1-0.8 | 2 |
| other centrally acting drugs | clonidine patch (Catapres- | 0.1-0.3 | 1 wkly |
| | TTS ®) | | |
| | methyldopa (Aldomet ®) | 250-1,000 | 2 |
| | reserpine (generic) | 0.1-0.25 | 1 |
| | guanfacine (Tenex ®) | 0.5-2 | 1 |
| Vasodilators | hydralazine (Apresoline ®) | 25-100 | 2 |
| | minoxidil (Loniten ®) | 2.5-80 | 1-2 |

In certain embodiments, the antihypertensive drug is administered in a subtherapeutic dose, as compared to the dosages set forth above.

### Pharmaceutical Compositions and Therapies

Administration of compounds useful within the invention may be achieved in a number of different ways, using methods known in the art. The therapeutic and prophylactic methods of the invention thus encompass the use of pharmaceutical compositions comprising the compounds useful within the invention to practice the methods of the invention. The pharmaceutical compositions of the invention may comprise (i) buntanetap, or the compound similar to buntanetap, (“compound (1)”) and (2) an antihypertensive agent (“compound (2)”) formulated and administered in a pharmaceutical formulation together with one or more pharmaceutically acceptable excipients.

Alternatively, the pharmaceutical compositions may be comprised of one of compounds (1) or (2) together with one or more pharmaceutically acceptable excipient. In such embodiments, compound (1) and (2) are administered separately but such that they provide overlapping therapeutic effects.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as, e.g., non-human primates, cattle, pigs, horses, sheep, cats, and dogs.

Typically, buntanetap dosages which may be administered in a method of invention to an animal, preferably a human, range in amount from 0.5 ug to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about lug to about 10 mg per kilogram of body weight of the animal. More preferably, the dosage will vary from about 3 μg to about 30 mg per kilogram of body weight of the animal.

Pharmaceutical compositions that are useful in the methods of invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses of, e.g., buntanetap and the high blood pressure medication. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The composition of the invention may consist of the active ingredient alone, in a form suitable for administration to a (human) subject or patient, or the composition may comprise at least one active ingredient and one or more pharmaceutically acceptable excipients.

In one embodiment, the compositions of the invention are formulated using one or more pharmaceutically acceptable carriers. Pharmaceutically acceptable carriers that are useful, include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The carrier may be a solvent or dispersion medium containing, for example, water, ethanol, polyol (for example, glycerol, propylene glycol, and liquid polyethylene glycol, and the like), suitable mixtures thereof, and vegetable oils.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined were desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an antioxidant and a chelating agent that inhibits the degradation of the compound. Preferred antioxidants for some compounds are BHT, BHA, alpha-tocopherol, and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing, or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredients in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Controlled- or sustained-release formulations of a composition of the invention may be made using conventional technology, in addition to the disclosure set forth elsewhere herein. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

Routes of administration of any of the compositions of the invention include oral, nasal, rectal, parenteral, sublingual, transdermal, transmucosal (e.g., sublingual, lingual, (trans) buccal, (trans) urethral, vaginal (e.g., trans-, and perivaginally), (intra) nasal, and (trans) rectal), intravesical, intrapulmonary, intraduodenal, intragastric, intrathecal, subcutaneous, intramuscular, intradermal, intra-arterial, intravenous, intrabronchial, inhalation, and topical administration.

For oral administration, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more inert, non-toxic pharmaceutically excipients. Such excipients include, for example an inert diluent such as lactose; granulating and disintegrating agents such as cornstarch; binding agents such as starch; and lubricating agents such as magnesium stearate. The oral compositions of the invention in the form of tablets or capsules prepared by conventional means with pharmaceutically acceptable excipients such as binding agents; fillers; lubricants; disintegrates; or wetting agents.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. For oral administration, if desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400).

Hard capsules comprising the active ingredients may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid preparation for oral administration may be in the form of solutions, syrups, or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters, or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications.

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patents Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, W O 98/11879, WO 97/47285, WO 93/18755, and WO 90/11757.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### DETAILED DESCRIPTION OF PREFERRED EMBODIMENTS

The following examples further illustrate aspects of the present invention. They are provided for the purpose of illustration only, and the invention is not limited to these examples, but rather encompasses all variations that are evident as a result of the teachings provided herein.

### Example 1

Three Phase I clinical studies have established buntanetap's safety. The pharmacokinetic analyses have demonstrated that the small lipophilic molecule readily enters the brain where its concentration is about 8 times higher than in plasma. Importantly, buntanetap normalized levels of APP, Tau, and αSYN in the cerebrospinal fluid (CSF) of MCI subjects at a dose of 4×60 mg/day. Buntanetap had a >12 h half-life in CSF of MCI subjects, and its effect in lowering these neurotoxic proteins and inflammation extended throughout the 12 h sampling period after the last dose (Maccecchini, et al., “Buntanetap (Posiphen) as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902). Therefore, we conclude that a much lower single daily buntanetap dose would be effective in the proposed study. In fact, much lower doses were studied in a double Alzheimer/Parkinson phase 2 study that was conducted and completed in 2021 (effective IND #72,654). The double phase 2 clinical trial recruited 14 AD (Alzheimer's disease) and 54 patients and treated them over 25 with a once daily dose of buntanetap. The 14 AD patients receive either 80 mg QD or placebo, whereas the 54 PD (Parkinson's disease) patients received 5, 10 20, 40 80 mg QD or placebo. In a nutshell the data shows that in AD and in PD patients (a) buntanetap crossed the blood brain barrier, (b) reduced neurotoxic protein biomarkers, (c) reduced inflammatory markers, (d) improved axonal and synaptic function, and most importantly improved the affected function in both patient populations. In AD patients, buntanetap improved cognition as measured by ADAS-Cog11 and WAIS coding speed (achieving statistical significance versus baseline at 80 mg dose but not placebo). In PD patients it improved at all doses motor function as measured by MDS-UPDRS (Part II, III, IV and total) with the maximum improvement for 10 & 20 mg and improved WAIS speed and accuracy (achieving statistical significance versus placebo in the 5 mg, 20 mg and 80 mg dose arms [p <0.05] of the broader study population [n=54] with the total for all doses also reporting statistically significant improvement [p <0.001]). The data demonstrates the potential benefits of reducing the overexpression of neurotoxic aggregating proteins on inflammation, axonal and synaptic function, and cognitive and functional health. We expect a larger sample population will allow buntanetap to fully demonstrate statistically significant cognitive and functional improvement resulting from the normalization of toxic protein levels in the next planned AD study as has already been demonstrated for PD (Fang et al., Prev Alz Dis 2022).

Buntanetap's effect on neurotoxic proteins. The drug lowers levels of APP in vitro in neuroblastoma cells (Mikillineni et al: Parkinson's Disease; Volume 2012, Article ID 142372, 13 pages. The Anticholinesterase Phenserine and Its Enantiomer Buntanetap as 5′ Untranslated- Region-Directed Translation Blockers of the Parkinson's Alpha Synuclein Expression). It also lowers levels of APP and all its fragments in APP/PS1 transgenic mice (A. F. Teich, et al., Alzheimer's & Dementia: Translational Research & Clinical Interventions 4 (2018) 37-45).

Buntanetap also lowers tau in vitro (Peter Davies Laboratory, Hofstra University, unpublished observation) and in vivo in Human tau mice (Peter Davies Laboratory, Hofstra University, unpublished observation). Buntanetap further lowers αSYN in vitro in neuroblastoma cells and in vivo in transgenic Parkinson's animals in the brain and in the gut (Kuo et. al. Am J Neurodegener Dis 2019; 8 (1): 1-15 www.AJND.us/ISSN: 216 591X/AJND0086080: Translational inhibition of α-synuclein by Buntanetap (posiphen) normalizes distal colon motility in transgenic Parkinson mice).

Buntanetap's efficacy in treating neurodegeneration in animal models: Restored memory and learning in an APP/PS1 transgenic (tg) mouse model of AD; Restored memory and learning in a Ts65dn mouse model of Down Syndrome (DS) [W. Mobley, UCSD, submitted 2020]; Preserved the retina in acute glaucoma [J Sundstrom; Hershey Medical School]; Restored colonic motility in a human SNCAA53T tg mouse model of PD (Kuo et. al., Am J Neurodegener Dis 2019; 8 (1): 1-15 www.AJND.us/ISSN: 216 591X/AJND0086080: Translational inhibition of α-synuclein by Buntanetap normalizes distal colon motility in transgenic Parkinson mice); Preserved memory and learning in traumatic brain injury rats (M-F Chesselet, submitted 2020).

Buntanetap's reversal of the neurotoxic cascade: buntanetap's mechanism of action is related to APP, Tau, and αSYN expression being regulated by IRP1 and by iron and the way these proteins contribute to neurodegeneration by accumulating as toxic aggregates that impair axonal transport and synaptic transmission, causing inflammation, and, finally, leading to nerve cell death (as described previously). By reducing APP, Tau, and αSYN levels, buntanetap treatment prevented this toxic cascade. In support of this hypothesis, it has been shown that buntanetap: Normalized anterograde and retrograde vesicle transport in fully differentiated Down syndrome nerve cells [Chen et al]; Normalized impaired synaptic transmission in rat striatum after traumatic brain injury (TBI) (M-F Chesselet; UCLA) and hippocampus of APP/PS1 tg mice; lowered inflammation in human CSF of MCI subjects and in the rat brain after TBI; protected nerve cells in rat substantia Ingra after TBI and in a rat acute glaucoma model (J Sundstrom; Hershey Medical School); reduced the levels of Huntingtin Protein through Translational Suppression (Chen, X.-Q.; Barrero, C. A.; Vasquez-Del Carpio, R.; Reddy, E. P.; Fecchio, C.; Merali, S.; Deglincerti, A.; Fang, C.; Rogers, J.; Maccecchini, M. L. Posiphen Reduces the Levels of Huntingtin Protein Through Translation Suppression. Pharmaceutics 2021, 13, 2109. https://doi.org/10.3390/pharmaceutics13122109); and proved to be safe and promising in both Alzheimer's and Parkinson's Patients (Fang et al., Prev Alz Dis 2022)).

The AD field has been dominated by approaches to prevent APP processing or remove Aβ in one of its many forms. These are downstream targets; buntanetap prevents the translational synthesis of the two main proteins involved in AD—APP and tau—and hence should remove all the downstream consequences produced by these proteins. Similarly, the PD field mostly focuses on inhibiting accumulation of αSYN aggregates and the effect of other proteins in this pathway, including LRRK or Parkin. Again, buntanetap prevents the synthesis of «SYN and thus it should stop the pathological cascade at the first step. Our data indicate that by normalizing the levels APP/Aβ, Tau/phospho-Tau, and αSYN, buntanetap normalizes axonal transport, lowers inflammation, and protects nerve cells from dying. (Mobley 2020, submitted for publication; Chesselet 2020, submitted for publication).

### Example 2A

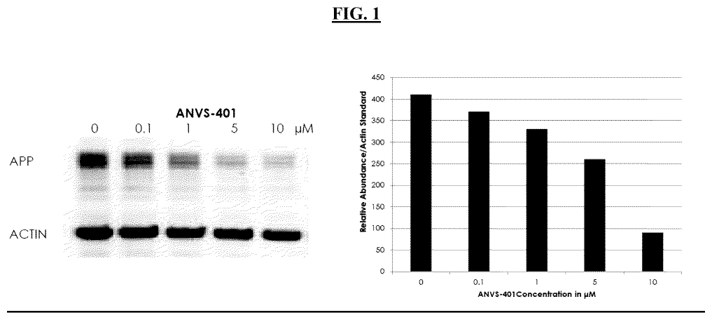

FIG. 1, APP in vitro. shows that buntanetap lowers APP in vitro in a dose-dependent manner in SH-SY-5Y human neuroblastoma cells. On the left is a Western blot showing buntanetap inhibition of APP in relation to Actin standard at concentrations of 0, 0.1, 1, 5 and 10 μM and a graph showing the same data plotted for statistical analysis purposes.

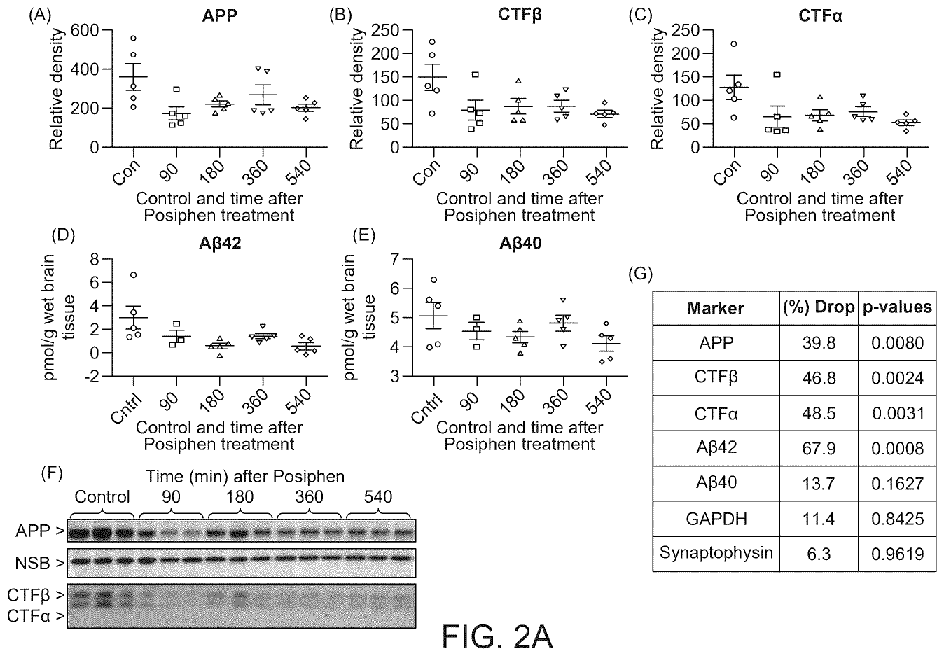

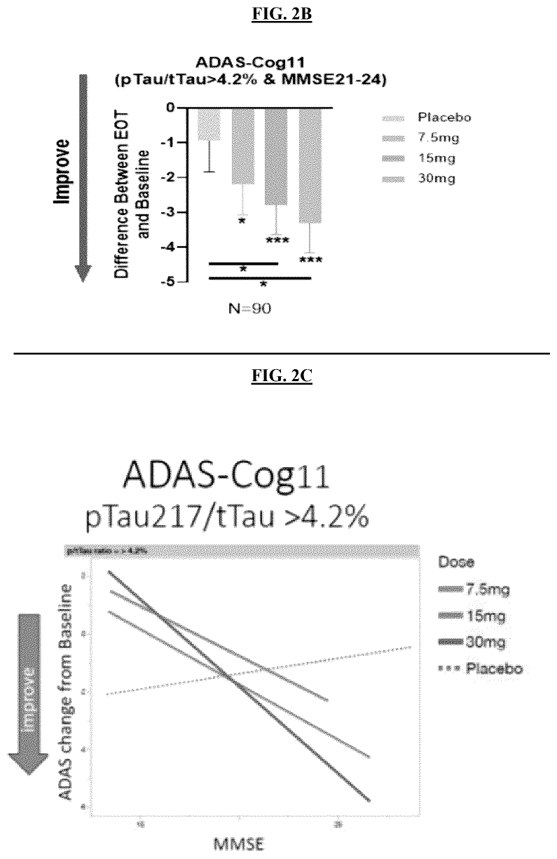

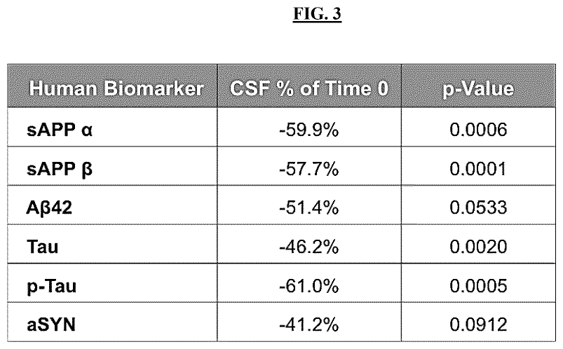

FIG. 2A, APP in vivo. This study was conducted to demonstrate the effect of buntanetap in inhibiting the translation of APP and its fragments in an AD model in vivo. The Table in FIG. 2 shows that buntanetap treatment of APP/PS1 transgenic AD mice reduced APP and its fragments in hippocampus. GAPDH and Synaptophysin were loading controls. FIG. 2 also includes a collection of graphs showing relative density of APP plotted against control and time after buntanetap treatment; relative density of CTFβ plotted against control and time after buntanetap treatment; relative density of CTFα plotted against control and time after buntanetap treatment; Aβ42 levels in brain tissue plotted against control and time after buntanetap treatment; and Aβ40 levels in brain tissue plotted against control and time after buntanetap treatment. Finally, FIG. 2 also includes a Western blot showing levels of APP, NSB, CTFβ, and CTFα after buntanetap treatment over time (minutes). In APP/PS1 mice expressing human mutations associated with familial AD, the data show that buntanetap treatment reduced APP and all related peptides in hippocampus for at least 9 hours after the last dose.

### Example 2B

A randomized, double-blind, placebo-controlled Phase II/III study trial investigating the efficacy, safety, and tolerability of buntanetap in patients with mild to moderate AD was conducted. This was a dose-ranging study where patients received either one of three doses of buntanetap (7.5 mg, 15 mg, or 30 mg) or placebo on top of their standard of care for 12 weeks. In this study, over 700 patients were screened, a total of 353 patients were enrolled, and 325 patients completed the study across 54 sites in the US. The study included mild to moderate AD patients whose Mini Mental State Examination (MMSE) scores at baseline ranged from 14 to 24. (www.clinicaltrials.gov (NCT05686044)).

Beyond safety, the trial assessed the changes in two co-primary endpoints: Alzheimer's Disease Assessment Scale-Cognitive Subscale 11 (ADAS-Cog 11) and Alzheimer's Disease Cooperative Study Clinician's Global Impression of Change (ADCS-CGIC), which assess cognition and activities of daily living. The study monitored for safety and collected plasma to measure several biomarkers to assess the disease state, potential disease progression, and treatment effects.

A significantly higher improvement in ADAS-Cog 11 scores in each treatment dose relative to placebo for patients with mild AD was observed. The analysis focused on biomarker-positive early AD patients (MMSE 21-24, pTau217/tTau≥4.2%) found that ADAS-Cog 11 was highly statistically significant at all 3 dose levels and in the combined dose levels compared to placebo as well as to baseline (FIG. 2B). The treatment response in the current study was not related to a patient's age or sex.

At the end of 3 months of treatment, placebo group demonstrated slight improvement (LSM(SE), 0.26 (0.91)), but not significantly different from baseline. However, all three buntanetap treatment groups showed statistically significant improvement from their corresponding baseline (7.5 mg improved 2.19 (0.87), p=0.013; 15 mg improved 2.79 (0.81), p=0.001; 30 mg improved 3.32 (0.82), P<0.001). Both 15 mg and 30 mg treatment groups also had a statistically significant improvement relative to placebo group (p=0.042 and 0.015 respectively). EOT-End of Treatment *P<0.05; **P<0.01; ***P<0.001.

When the baseline MMSE scores for patients positive for AD according to their pTau217/tTau >4.2% ratio were subdivided, a dose-dependent relationship to MMSE at baseline was observed. It was concluded that the response to buntanetap treatment is more pronounced in mild AD patients than in those with more advanced AD. The response in the 30 mg dose treatment group R2=0.17 (R² or the coefficient of determination), p <0.001, indicates statistical significance of the MMSE score, which was not evident in the placebo group. FIG. 2C confirms the efficacy of buntanetap as previously shown in FIG. 2B.

It was concluded that was a three-fold difference in the proportion of participants who improved in the 30 mg group relative to placebo (Table 1A).

TABLE 1A
Responders vs Non-Responders

| | Responders | | Non-Responders | | |
|---|---|---|---|---|---|
| | (Number & | | (Number & | | Total |
| Dose | Percentage) | | Percentage) | | Number |
| Placebo | 6 | (27.27%) | 16 | (72.73%) | 22 |
| 7.5 mg Buntanetap | 14 | (73.68%)** | 5 | (26.32%) | 19 |
| 15 mg Buntanetap | 18 | (72.00%)** | 7 | (28.00%) | 25 |
| 30 mg Buntanetap | 21 | (87.50%)*** | 3 | (12.50%) | 24 |

**Contrast with Placebo, p value < 0.01,
***Contrast with Placebo, p value < 0.001

It was concluded that the data presented in FIGS. 2B and 2C demonstrates efficacy of buntanetap in early AD patients.

### Example 3

FIG. 3 is a study of subjects with mild cognitive impairment (MCI) for early proof of mechanism (POM) using a well-tolerated dose of buntanetap. Before and after 10 days of buntanetap administration to the MCI subjects, plasma, and cerebrospinal fluid (CSF) samples were obtained for analysis of levels of secreted(s) APPα and APPβ, and Aβ42, Tau (total and phosphorylated), and inflammatory markers. FIG. 3 shows the reduction of APP/Aβ, tau/phosphor-tau and alpha-synuclein in the spinal fluid of mildly cognitive impaired patients. In this study, buntanetap normalized these aggregating proteins in CSF of MCI subjects in accordance with the data seen in animals.

### Example 4

A study has established association of terazosin, doxazosin, or alfuzosin and reduced risk of dementia with Lewy bodies (“DLB”). Hart et al., Association of Terazosin, Doxazosin, or Alfuzosin Use and Risk of Dementia With Lewy Bodies in Men, Neurology, Jul. 23, 2024 issue. The study analyzed data from more than 600,000 men.

The study reported that men who were newly started on terazosin, doxazosin, or alfuzosin had a lower hazard of developing DLB than matched men taking tamsulosin (n=242,716, 728,256 person-years, hazard ratio [HR] 0.60, 95% CI 0.50-0.71) or 5ARI (n=130,872, 399,316 person-years, HR 0.73, 95% CI 0.57-0.93), while the hazard in men taking tamsulosin was similar to that of men taking 5ARI (n=159,596, 482,280 person-years, HR 1.17, 95% CI 0.96-1.42). “Specifically, the risk was 40% lower compared to tamsulosin and 37 percent lower compared to 5ARIs.” Tsai, Prostate Meds Slash Dementia Risk by up to 40 Percent: Study, Epoch Health, Jun. 26, 2024.

The study provides Class III evidence that terazosin, doxazosin, or alfuzosin use reduces the rate of developing DLB in adult men.

While the invention has been disclosed with reference to specific embodiments, it is apparent that other embodiments and variations of this invention may be devised by others skilled in the art without departing from the true spirit and scope of the invention. All patents and publications cited herein are incorporated by reference in their entirety. The appended claims are intended to be construed to include all such embodiments and equivalent variations.

What is claimed is:

1. A method for treating a neurodegenerative disease comprising co-administration to a human in need thereof:
(i) an amount of a compound selected from the group consisting of Formula (I), Formula (II) and Formula (III): 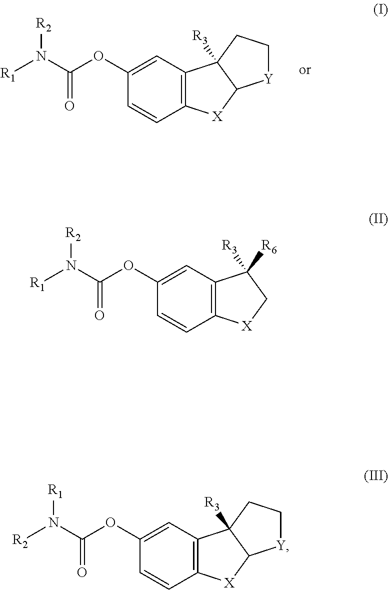
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(ii) an amount of an antihypertensive agent.

2. The method of claim 1, wherein the antihypertensive agent is an alpha-adrenergic blocker.

3. The method of claim 2, wherein the alpha-adrenergic blocker is selected from a group consisting of terazosin, doxasozin, alfuzocin, and pharmaceutically acceptable salts thereof.

4. The method of claim 3, wherein the compound selected from the group consisting of Formula (I), Formula (II) and Formula (III) is buntanetap or a pharmaceutically acceptable salt thereof.

5. The method of claim 1, wherein buntanetap or the pharmaceutically acceptable slat thereof and the antihypertensive agent or the pharmaceutically acceptable salt thereof are administered in the same formulation, and the formulation is administered orally, parenterally, intravenously, subcutaneously, sublingually, via suppository, nasally, topically, transdermally, or via an implant under the skin.

6. The method of claim 1, wherein the formulation is chronically administered to a human who is experiencing hypertension.

7. The method of claim 1, wherein the human is not demonstrating symptoms of a neurodegenerative disease.

8. The method of claim 3, wherein buntanetap or the pharmaceutically acceptable salt thereof is administered orally once-a-day in an amount from about 0.1 mg to about 0.9 mg.

9. A method for treating a neurodegenerative disease in a human in need thereof comprising administering to the human a dosage form comprising buntanetap or a pharmaceutically acceptable salt thereof and an alpha-adrenergic blocker.

10. The method of claim 9, wherein the alpha-adrenergic blocker selected from the group consisting of terazosin, doxasozin, alfuzocin, and pharmaceutically acceptable salts thereof.

11. The method of claim 9, wherein buntanetap or the pharmaceutically acceptable salt thereof is administered orally once-a-day in an amount from about 0.1 mg to about 0.9 mg.

12. A pharmaceutical composition, comprising
(i) an amount of a compound selected from the group consisting of Formula (I), Formula (II) and Formula (III): 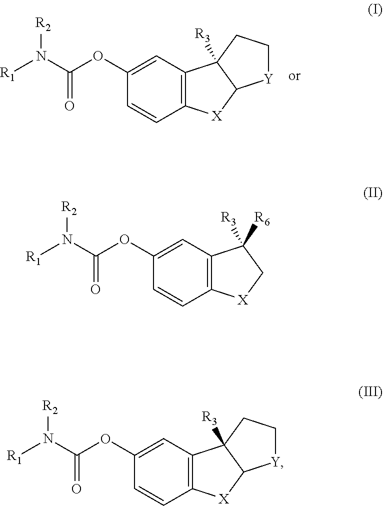
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof;
(ii) an amount of compound which is an alpha-adrenergic blocker; and
(iii) at least one pharmaceutically acceptable excipient.

13. The pharmaceutical composition of claim 12, wherein the compound selected from the group consisting of Formula (I), Formula (II) and Formula (III) is buntanetap or a pharmaceutically acceptable salt thereof.

14. The pharmaceutical composition of claim 13, wherein the alpha-adrenergic blocker is selected from the group consisting of terazosin, doxasozin, alfuzocin, and pharmaceutically acceptable salts thereof.

15. The pharmaceutical composition of claim 14, which is a tablet.

16. The method of claim 1, wherein the neurodegenerative disease is selected from the group consisting of Alzheimer's disease, chronic traumatic encephalopathy, frontotemporal dementia, Parkinson's disease, an alpha-synucleopathy, Prion's disease, Down Syndrome, Huntington's disease, Amyloid Lateral Sclerosis, and multiple sclerosis.

17. The method of claim 16, wherein the neurodegenerative disease is Alzheimer's disease.

18. The method of claim 16, wherein the neurodegenerative disease is Parkinson's disease.
